SEQUENCE LISTING (28 sequences)

SEQ ID NO: 1 - protein, 129 aa, F. nucleatum
MKKFLLLAVLAVSASAFAANDAASLVGELQALDAEYQNLANQEEARFNEERAQADAARQALAQNEQVYNELSQRAQRLQAEANTRFYKSQYQDLASKYEDALKKLESEMEQQKAIISDFEKIQALRAGN

SEQ ID NO: 2 - protein, 111 aa, F. nucleatum
ANDAASLVGELQALDAEYQNLANQEEARFNEERAQADAARQALAQNEQVYNELSQRAQRLQAEANTRFYKSQYQDLASKYEDALKKLESEMEQQKAIISDFEKIQALRAGN

SEQ ID NO: 3 - protein, 120 aa, F. nucleatum
MANDAASLVGELQALDAEYQNLANQEEARFNEERAQADAARQALAQNEQVYNELSQRAQRLQAEANTRFYKSQYQDLASKYEDALKKLESEMEQQKAIISDFEKIQALRAGNGGHHHHHH

SEQ ID NO: 4 - protein, 112 aa, F. nucleatum
MANDAASLVGELQALDAEYQNLANQEEARFNEERAQADAARQALAQNEQVYNELSQRAQRLQAEANTRFYKSQYQDLASKYEDALKKLESEMEQQKAIISDFEKIQALRAGN

SEQ ID NO: 5 - protein, 44 aa, F. nucleatum
QVYNELSQRAQRLQAEANTRFYKSQYQDLASKYEDALKKLESEM

SEQ ID NO: 6 - protein, 47 aa, F. nucleatum
MAPQVYNELSQRAQRLQAEANTRFYKSQYQDLASKYEDALKKLESEM

SEQ ID NO: 7 - protein, 55 aa, F. nucleatum
MAPQVYNELSQRAQRLQAEANTRFYKSQYQDLASKYEDALKKLESEMGGHHHHHH

SEQ ID NO: 8 - protein, 18 aa, F. nucleatum
MKKFLLLAVLAVSASAFA

SEQ ID NO: 9 - DNA, 366 bp, F. nucleatum
atggcgaatgatgccgccagtctggttggcgaactgcaagcgctggatgccgagtaccagaatctggcgaatcaagaagaggcccgcttcaatgaagaacgtgcccaagcggatgccgcccgccaagcgctggcccagaacgagcaagtgtacaacgagctcagccaacgtgcccaacgtctgcaagccgaggccaatacccgcttctacaagagtcagtaccaagatctggccagtaagtacgaggacgcgctgaagaaactggaaagcgagatggagcagcagaaggcgatcatcagcgatttcgagaagatccaagccctccgcgcgggtaatggcggccaccaccatcatcaccattaataa

SEQ ID NO: 10 - DNA, 342 bp, F. nucleatum
atggcgaatgatgccgccagtctggttggcgaactgcaagcgctggatgccgagtaccagaatctggcgaatcaagaagaggcccgcttcaatgaagaacgtgcccaagcggatgccgcccgccaagcgctggcccagaacgagcaagtgtacaacgagctcagccaacgtgcccaacgtctgcaagccgaggccaatacccgcttctacaagagtcagtaccaagatctggccagtaagtacgaggacgcgctgaagaaactggaaagcgagatggagcagcagaaggcgatcatcagcgatttcgagaagatccaagccctccgcgcgggtaattaataa

SEQ ID NO: 11 - DNA, 171 bp, F. nucleatum
atggccccacaagtgtacaacgagctcagccaacgtgcccaacgtctgcaagccgaggccaatacccgcttctacaagagtcagtaccaagatctggccagtaagtacgaggacgcgctgaagaaactggaaagcgagatgggcggccaccaccatcatcaccattaataa

SEQ ID NO: 12 - DNA, 147 bp, F. nucleatum
atggccccacaagtgtacaacgagctcagccaacgtgcccaacgtctgcaagccgaggccaatacccgcttctacaagagtcagtaccaagatctggccagtaagtacgaggacgcgctgaagaaactggaaagcgagatgtaataa

SEQ ID NO: 13 - protein, 160 aa, Haemophilus influenzae
MKKIILTLSLGLLTACSAQIQKAKQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 14 - protein, 129 aa, Fusobacterium nucleatum
MKKFLLLAVLAVSASAFAANDAASLVGELQALDAEYQNLANQEEARFNEEKAQADAAKQALAQNEQVYNELSQRAQRLQAEANTRFYKSQYQELASKYEDALKKLEAEMEQQKGVISDFEKIQALRAGN

SEQ ID NO: 15 - protein, 111 aa, Fusobacterium nucleatum
ANDAASLVGELQALDAEYQNLANQEEARFNEEKAQADAAKQALAQNEQVYNELSQRAQRLQAEANTRFYKSQYQELASKYEDALKKLEAEMEQQKGVISDFEKIQALRAGN

SEQ ID NO: 16 - protein, 112 aa, Fusobacterium nucleatum
MANDAASLVGELQALDAEYQNLANQEEARFNEEKAQADAAKQALAQNEQVYNELSQRAQRLQAEANTRFYKSQYQELASKYEDALKKLEAEMEQQKGVISDFEKIQALRAGN

SEQ ID NO: 17 - protein, 120 aa, Fusobacterium nucleatum
MANDAASLVGELQALDAEYQNLANQEEARFNEEKAQADAAKQALAQNEQVYNELSQRAQRLQAEANTRFYKSQYQELASKYEDALKKLEAEMEQQKGVISDFEKIQALRAGNGGHHHHHH

SEQ ID NO: 18 - protein, 129 aa, Fusobacterium nucleatum
MKKFLLLAVLVVSASAFAANDAASLVGELQALDAEYQNLANQEEARFNEEKAQADAAKQALAQNEQVYNELSQRAQRLQAEANTRFYKSQYQDLASKYEDALKKLEAEMEQQKGVISDFEKIQALRAGN

SEQ ID NO: 19 - protein, 111 aa, Fusobacterium nucleatum
ANDAASLVGELQALDAEYQNLANQEEARFNEEKAQADAAKQALAQNEQVYNELSQRAQRLQAEANTRFYKSQYQDLASKYEDALKKLEAEMEQQKGVISDFEKIQALRAGN

SEQ ID NO: 20 - protein, 112 aa, Fusobacterium nucleatum
MANDAASLVGELQALDAEYQNLANQEEARFNEEKAQADAAKQALAQNEQVYNELSQRAQRLQAEANTRFYKSQYQDLASKYEDALKKLEAEMEQQKGVISDFEKIQALRAGN

SEQ ID NO: 21 - protein, 120 aa, Fusobacterium nucleatum
MANDAASLVGELQALDAEYQNLANQEEARFNEEKAQADAAKQALAQNEQVYNELSQRAQRLQAEANTRFYKSQYQDLASKYEDALKKLEAEMEQQKGVISDFEKIQALRAGNGGHHHHHH

SEQ ID NO: 22 - protein, 129 aa, Fusobacterium nucleatum
MKKFLLLAVLAVSASAFAATDAASLVGELQALDAEYQNLANQEEARFNEERAQADAARQALAQNEQVYNELSQRAQRLQAEANTRFYKSQYQELASKYEDALKKLEAEMEQQKAVISDFEKIQALRAGN

SEQ ID NO: 23 - protein, 111 aa, Fusobacterium nucleatum
ATDAASLVGELQALDAEYQNLANQEEARFNEERAQADAARQALAQNEQVYNELSQRAQRLQAEANTRFYKSQYQELASKYEDALKKLEAEMEQQKAVISDFEKIQALRAGN

SEQ ID NO: 24 - protein, 112 aa, Fusobacterium nucleatum
MATDAASLVGELQALDAEYQNLANQEEARFNEERAQADAARQALAQNEQVYNELSQRAQRLQAEANTRFYKSQYQELASKYEDALKKLEAEMEQQKAVISDFEKIQALRAGN

SEQ ID NO: 25 - protein, 120 aa, Fusobacterium nucleatum
MATDAASLVGELQALDAEYQNLANQEEARFNEERAQADAARQALAQNEQVYNELSQRAQRLQAEANTRFYKSQYQELASKYEDALKKLEAEMEQQKAVISDFEKIQALRAGNGGHHHHHH

SEQ ID NO: 26 - protein, 44 aa, Fusobacterium nucleatum
QVYNELSQRAQRLQAEANTRFYKSQYQELASKYEDALKKLEAEM

SEQ ID NO: 27 - protein, 44 aa, Fusobacterium nucleatum
QVYNELSQRAQRLQAEANTRFYKSQYQDLASKYEDALKKLEAEM

SEQ ID NO: 28 - protein, 44 aa, Fusobacterium nucleatum; identical to SEQ ID NO: 26

(19) United States
(12) Patent Application Publication
(10) Pub. No.: US 2023/0293659 A1
(43) Pub. Date: Sep. 21, 2023

(54) TRUNCATED FUSOBACTERIUM NUCLEATUM FUSOBACTERIUM ADHESIN A (FADA) PROTEIN AND IMMUNOGENIC COMPOSITIONS THEREOF

(71) Applicant: GlaxoSmithKline Biologicals SA, Rixensart (BE)

(72) Inventors: Cindy CASTADO, Rixensart (BE); Sandra GIANNINI, Rixensart (BE)

(21) Appl. No.: 18/006,627

(22) PCT Filed: Jul. 30, 2021
(86) PCT No.: PCT/EP2021/071383

§ 371 Date: Jan. 24, 2023

Related U.S. Application Data
(60) Provisional application No. 63/060,264, filed on Aug. 3, 2020.

(51) Int. Cl. A61K 39/114; A61P 37/04

(52) U.S. Cl. CPC A61K 39/114; A61P 37/04; A61K 2039/53

(57) ABSTRACT

The application discloses a truncated Fusobacterium nucleatum Fusobacterium adhesin A (FadA) protein wherein at least a signal peptide which is at least 80%, 85%, 90% or 95% identical to SEQ ID NO:8 is deleted from the N-terminus of the FadA protein. Polynucleotides and vectors encoding the truncated FadA protein and bacteriophage comprising a gene encoding FadA as well of methods of treating or preventing disease such as colorectal cancer or periodontitis are also disclosed in the application.

### TECHNICAL FIELD

The present invention relates to the field of immunogenic compositions and vaccines directed against Fusobacterium nucleatum, particularly those comprising a FadA antigen, or a polynucleotide encoding a FadA antigen and vectors encoding a FadA antigen. The invention also encompasses the use of such FadA antigens, immunogenic compositions, vaccine and polynucleotides encoding FadA in the treatment or prevention of human disease including colorectal cancer and peridontitis.

### BACKGROUND

Fusobacterium nucleatum is an oral Gram-negative anaerobic bacterium, indigenous to the human oral cavity, but absent or infrequently detected elsewhere in the body under normal conditions (Ioannis Koliarakis, Ippokratis Messaritakis, Taxiarchis Konstantinos Nikolouzakis, George Hamilos, John Souglakos and John Tsiaoussis; Oral Bacteria and Intestinal Dysbiosis in Colorectal Cancer; Int. J. Mol. Sci. 2019, 20, 4146).

The bacterial species of the oral microbiota mainly coexist by forming complex polymicrobial communities. In this symbiotic state of co-aggregation, the various bacterial species maintain the homeostasis of the oral ecosystem, creating a balance between pathogens and commensals. Any alteration in the conditions (resulting in a disruption of this balance) caused by either internal (e.g., genetics) or external (e.g., diet, smoking, toxicants, antibiotics) factors could enhance the pathogenetic potential of the oral microbiota, furthering the progression of oral diseases. (Dowd, S. E.; Wolcott, R. D.; Sun, Y.; McKeehan, T.; Smith, E.; Rhoads, D. Polymicrobial nature of chronic diabetic foot ulcer biofilm infections determined using bacterial tag encoded FLX amplicon pyrosequencing (bTEFAP). PLoS ONE 2008, 3, e3326)

Under disease conditions, F. nucleatum is one of the most prevalent species found in oral and extra-oral sites, commonly recovered from different monomicrobial and mixed infections in humans and animals. (Alex B. Berezow, R. P. D. (2011). “Microbial Shift and Periodontitis.” Periodontol 2000).

F. nucleatum is a heterogeneous species with five proposed subspecies (ss), i.e. ss animalis, ss fusiforme, ss nucleatum, ss polymorphum, and ss vincentii, whose prevalence in disease vary. F. nucleatum is one of the most abundant species in the oral cavity, in both diseased and healthy individuals. (Elisabeth M Bik; Bacterial diversity in the oral cavity of 10 healthy individuals; The ISME Journal (2010) 4, 962-974). It is implicated in various forms of periodontal diseases including the mild reversible form of gingivitis and the advanced irreversible forms of periodontitis including chronic periodontitis, localized aggressive periodontitis and generalized aggressive periodontitis. It is also frequently associated with endodontic infections such as pulp necrosis and periapical periodontitis. The prevalence of F. nucleatum increases with the severity of disease, progression of inflammation and pocket depth. Among the five subspecies, ss fusiforme and ss vincentii are more frequently associated with health while ss nucleatum with disease (Lourenco T G, Heller D, Silva-Boghossian C M, Cotton S L, Paster B J, Colombo A P. J Clin Periodontol. 2014; 41:1027-1036, Gharbia S E, Shah H N, Lawson P A, Haapasalo M. Oral Microbiol Immunol. 1990; 5:324-327. In addition to the periodontal sites, F. nucleatum is detected in saliva, with its quantities increased in patients with gingivitis and periodontitis, compared to the healthy controls. Serum antibody titers to F. nucleatum have been reported to be elevated in diseased patients (Saygun I, Nizan N et al—Salivary infectious agents and periodontal disease status J. Periodontal Res (2011) 46: 235-239). The abundance of F. nucleatum is affected by environmental factors. Animal studies support a causative role of F. nucleatum in periodontal infections. Mono-infection of mice with F. nucleatum induces periodontal bone loss or abscess. When F. nucleatum is co-infected with other oral species, e.g. Tannerella forsythia, Porphyromonas gingivalis and Streptococci, respectively, synergy in virulence is observed as evidenced by enhanced bone loss, abscess, or death.

In addition to its role in oral inflammation and disease F. nucleatum is also associated with cancer, including colorectal cancer, and with premature births and term stillbirths [Han Y W, Redline R W, Li M, Yin L, Hill G B, McCormick T S. Infect Immun. 2004; 72:2272-2279, Krejs G J. Dig Dis. 2010; 28:355-358]. In addition, F. nucleatum is closely connected with liver abscess [Ahmed Z, Bansal S K, Dhillon S. World J Gastroenterol. 2015; 21:3731-3735; Yoneda M, Kato S, Mawatari H, Kirikoshi H, Imajo K, Fujita K, Endo H, Takahashi H, Inamori M, Kobayashi N, et al. Hepatol Res. 2011; 41:194-196], appendicitis and infections of the head and neck, including mastoiditis, tonsillitis and maxillary sinusitis [Yarden-Bilaysky H, Raveh E, Livni G, Scheuerman O, Amir J, Bilaysky E. Int J Pediatr Otorhinolaryngol. 2013; 77:92-96; Salö M, Marungruang N, Roth B, Sundberg T, Stenström P, Arnbjornsson E, Fåk F, Ohlsson B. Int J Colorectal Dis. 2017; 32:19-28]. Increasing evidence has indicated that the levels of F. nucleatum are significantly elevated in tumor tissues and stool specimens of colorectal cancer (CRC) patients relative to those in normal controls [Castellarin M, Warren R L, Freeman J D, Dreolini L, Krzywinski M, Strauss J, Barnes R, Watson P, Allen-Vercoe E, Moore R A, et al. Fusobacterium nucleatum infection is prevalent in human colorectal carcinoma. Genome Res. 2012; 22:299-306; McCoy A N, Araújo-Pérez F, Azcárate-Peril A, Yeh J J, Sandler R S, Keku T O. PLoS One. 2013; 8:e53653, Yu-Yuan Li, Quan-Xing Ge, Jie Cao, Yong-Jian Zhou, Yan-Lei Du, Bo Shen, Yu-Jui Yvonne Wan, and Yu-Qiang Nie World J Gastroenterol. 2016 Mar. 21; 22(11): 3227-3233.]. Researchers have reported that F. nucleatum may contribute to the development of CRC and that it is considered to be a potential risk factor for CRC progression [Flanagan L, Schmid J, Ebert M, Soucek P, Kunicka T, Liska V, Bruha J, Neary P, Dezeeuw N, Tommasino M, et al. Eur J Clin Microbiol Infect Dis. 2014; 33:1381-1390; Rubinstein M R, Wang X, Liu W, Hao Y, Cai G, Han Y W. Cell Host Microbe. 2013; 14:195-206]. Investigators have demonstrated that a higher abundance of F. nucleatum in CRC is associated with a shorter survival time [Mima K, Nishihara R, Qian Z R, Cao Y, Sukawa Y, Nowak J A, Yang J, Dou R, Masugi Y, Song M, et al. Gut. 2016; 65:1973-1980, Andrew T. Kunzmann, Marcela Alcântara Proença, Haydee W T Jordao, Katerina Jiraskova, Michaela Schneiderova, Miroslav Levy, Václav Liska, Tomas Buchler, Ludmila Vodickova, Veronika Vymetalkova, Ana Elizabete Silva, Pavel Vodicka & David J. Hughes, European Journal of Clinical Microbiology & Infectious Diseases volume 38, pages 1891-1899 (2019), Ana Carolina de Carvalho, Leandro de Mattos Pereira, José Guilherme Datorre, Wellington Dos Santos, Gustavo Noriz Berardinelli, Marcus de Medeiros Matsushita, Marco Antonio Oliveira, Ronilson Oliveira Duraes, Denise Peixoto Guimãraes, Rui Manuel Reis Front Oncol 2019 Aug. 29; 9:813. TaChung Yu, Fangfang Guo, Yanan Yu, Tiantian Sun, Dan Ma, Jixuan Han, Yun Qian, Ilona Kryczek, Danfeng Sun, Nisha Nagarsheth, Yingxuan Chen, Haoyan Chen, Jie Hong, Weiping Zou, Jing-Yuan Fang Cell. 2017 Jul. 27; 170(3):548-563]. Several researchers have also shown that a high-abundance of F. nucleatum is associated with specific tumor molecular events, including CpG island methylator phenotype (CIMP), microsatellite instability (MSI), and genetic mutations in BRAF, CHD7, CHD8 and TP53 [Mima K, Nishihara R, Qian Z R, Cao Y, Sukawa Y, Nowak J A, Yang J, Dou R, Masugi Y, Song M, et al. Gut. 2016; 65:1973-1980; Tahara T, Yamamoto E, Suzuki H, Maruyama R, Chung W, Garriga J, Jelinek J, Yamano H O, Sugai T, An B, et al. Cancer Res. 2014; 74:1311-1318]. However, F. nucleatum was previously regarded as a passenger bacterium in human intestinal tract [Tjalsma H, Boleij A, Marchesi J R, Dutilh B E. Nat Rev Microbiol. 2012; 10:575-582; Allen-Vercoe E, Strauss J, Chadee K. Gut Microbes. 2011; 2:294-298]. Recently, it has been considered to be a potential promoter of CRC susceptibility [McCoy A N, Araújo-Pérez F, Azcárate-Peril A, Yeh J J, Sandler R S, Keku T O. PLoS One. 2013; 8:e53653; Tahara T, Yamamoto E, Suzuki H, Maruyama R, Chung W, Garriga J, Jelinek J, Yamano H O, Sugai T, An B, et al. Cancer Res. 2014; 74:1311-1318]. Kostic et al have confirmed (Kostic A D, Chun E, Robertson L, Glickman J N, Gallini C A, Michaud M, Clancy T E, Chung D C, Lochhead P, Hold G L, et al. Cell Host Microbe. 2013; 14:207-215) that F. nucleatum promotes colorectal tumorigenesis in Apcm^{min/+} mice. Rubinstein et al [Rubinstein M R, Wang X, Liu W, Hao Y, Cai G, Han Y W. Cell Host Microbe. 2013; 14:195-206] have reported that F. nucleatum stimulates tumor cell growth in CRC by activating β-catenin signaling and inducing oncogenic gene expression via the FadA adhesion virulence factor. Together, these studies show that F. nucleatum plays an important role in the promotion of CRC by stimulating tumor cell growth in additon to activating inflammatory pathways supporting that F. nucleatum plays an active role in CRC progression rather than being a consequence of tumor progression. F. nucleatum subspecies nucleatum and animalis have been associated with CRC progression.

Fusobacterium adhesin A (FadA) is a novel small and highly conserved adhesin containing 129 amino acids. FadA is composed of two forms. Pre-FadA is a non secreted form containing a signal peptide whereas mature FadA (mFadA) is the secreted form. The functional FadA complex (FadAc) is made up of both forms. FadA is involved in bacterial attachment to host cells and invasion. A FadA-deleted mutant is defective in host-cell attachment and invasion as well as colonization of murine placenta. However, a complementation with FadA restores host-cell adhesion, invasion and colonization of the placenta (S. Témoin et al., 2012, FEBS Lett. Vol. 586; 1-6). FadA has been shown to be essential for adherence, invasion and activation of oncogenic/inflammatory pathways in colorectal cancer cell lines (Rubinstein M R, Wang X, Liu W, Hao Y, Cai G, Han Y W. Cell Host Microbe. 2013; 14:195-206).

It is proposed that FadA forms filaments in the following way. MFadA is secreted across the inner membrane and self assembles in the periplasm. As more mFadA is synthesized and secreted, this leads to elongation of the FadA chain which extends across the outer membrane. When elongation of the filament stops, preFadA is incorporated onto the base. It is envisaged that the N-terminal part of pre-FadA forms a short helix connected to the mFadA helix via a hairpin to form interhelical contacts potentially mediated via Leu residues on both helices. The N-terminal helical hairpin could serve as an anchor in the inner membrane (S. Témoin et al., 2012 Febs Lett. 586; 1-6).

Suggested vaccine candidates for use against F. nucleatum associated disease include the use of UV inactivated F. nucleatum against periodonal disease (Liu P F, Hake 3K, Gallo R L, Huang C M. Liu P F, et al. Vaccine 2009 Mar. 4; 27(10)1589-95), the use of fusion protein FlaB-tFomA against periodontal disease (Puth S, et al. Mucosal Immunol. 2019 March; 12(2):565-579); and FomA porin for treatment of periodontal disease (Liu P F et al Vaccine. 2010 Apr. 26; 28(19):3496-505).

There remains an unmet medical need to provide remedies for the pathologies involving Fusobacterium nucleatum, including treatment of periodontitis and cancers including colorectal cancer. The inventors herein propose a new approach to the treatment or prevention of F. nucleatum associated disease involving the provision of a FadA immunogen.

### SUMMARY OF THE INVENTION

The present inventors propose a truncated FadA as an immunogen. The truncated FadA is missing at least the N-terminal signal peptide, for example at least 10, 12, 14, 16 or 18 amino acids from the N-terminus of FadA according to SEQ ID NO:1 or analogous sequences.

Accordingly, the present invention provides a truncated Fusobacterium nucleatum Fusobacterium adhesin A (FadA) protein wherein at least a signal peptide which is at least 80%, 85%, 90% or 95% identical to SEQ ID NO:8 is deleted from the N-terminus of the FadA protein or a truncated Fusobacterium nucleatum Fusobacterium adhesin A (FadA) protein having a truncation at the N-terminus such that at least 14, 15, 16, 17 or 18 of the amino acids at the N-terminus of SEQ ID NO:1 are not present.

The proposed FadA immunogen includes the tip region of FadA (for example, comprising amino acids corresponding to residues 84-87 of SEQ ID NO:1) which mediates attachment to human cells. Such FadA immunogens are optionally capable of generating an immune response which blocks the binding of FadA to cadherins. Further potential advantages of the FadA immunogens include a decrease in aggregation of FadA in comparison to the full length FadA leading to the facilitation of production and purification.

In a further embodiment, the present invention provides a polynucleotide encoding the recombinant FadA of the invention.

In a further embodiment, the present invention provides a vector comprising the polynucleotide of the invention under the control of a promoter.

In a further embodiment, the present invention provides a recombinant bacteriophage comprising a phage genome polynucleotide including a gene encoding the recombinant FadA protein of the invention or the polynucleotide of the invention.

In a further embodiment, the present invention provides a recombinant bacteriophage genome polynucleotide comprising a gene encoding Fusobacterium adhesin A (FadA) according to the invention or the polynucleotide of the invention.

In a further embodiment, the present invention provides a pharmaceutical composition comprising the truncated FadA protein of the invention, the polynucleotide of the invention, the vector of the invention, the recombinant bacteriophage of the invention or the recombinant bacteriophage genome polynucleotide of the invention.

In a further embodiment, the present invention provides a vaccine comprising the truncated FadA protein of the invention, the polynucleotide of the invention, the recombinant bacteriophage of the invention or the recombinant bacteriophage genome polynucleotide of the invention.

In a further embodiment, the present invention provides a truncated FadA protein of the invention, a polynucleotide of the invention, the recombinant bacteriophage of the invention or the recombinant bacteriophage genome polynucleotide of the invention for use in therapy (for example in the treatment or prevention of CRC, for example in humans or the treatment or prevention of periodontitis, for example in humans).

In a further embodiment, the present invention provides a method of treatment of colorectal cancer comprising the step of: a) administering the truncated FadA protein of the invention or the recombinant bacteriophage of the invention or the recombinant bacteriophage genomic polynucleotide of the invention, to a patient in need thereof.

In a further embodiment, the present invention provides a method of treatment of periodontitis comprising the step of: a) administering the truncated FadA protein of the invention or the recombinant bacteriophage of the invention or the recombinant bacteriophage genomic polynucleotide of the invention, to a patient in need thereof.

### DESCRIPTION OF FIGURES

FIG. 1: Panel A—Sequence of Fusobacterium nucleatum subsp. nucleatum strain ATCC 25586. The annotation is: signal peptide (SP); amino acids 1-18/Coiled-coils structure: 22-81 and 88-122/mFadA starts after SP (in yellow highlight, which is not cleaved in pre-FadA)/Central hairpin residues (red lettering)/Leucine (Leu or L) residues in green or cyan highlight: leu-leu contacts stabilizing the 2 antiparallel helices of one monomer (L53-L84 and L60-L76 Crystal Structure of FadA Adhesin from Fusobacterium nucleatum Reveals a Novel Oligomerization Motif, the Leucine Chain” S. Nithianantham et al., 2009, The J. of Biological Chemistry vol. 284(6)) Tyr: Tyrosine (Tyr or Y, in grey highlight) aromatic rings are buried to provide hydrophobic contact with Leu and other apolar side chains (Y50, Y73 and Y80) Leu residues involved in non-covalent interaction with other monomers (numbering on mFadA) in pink highlight in one monomer, underlined on the other one: L7, 11, 14 and 21 with L53, 76 and 84
additional intermolecular interaction: L14, L21, L60 and L76 of one monomer with Y80, Y73, Y18 of the other one, respectively
Intermolecular salt-bridges: E10 (with R/K86), D15 (with R56), E17 (with K79), E25, R33, R56, R67, E75, K79 and R86 (see in literature information section for more information+predicted structure of FadA Oligomer)
L14 and L76: critical for biological function

Panel B—Model demonstrating the tertiary structure of FadA (PDB code: 3ETW)

FIG. 2: Comparison of the sequence of FadA from five strains of F. nucleatum; 12230, ATCC25586, ChDC317, ATCC23726 and ChDCF316. Black shading means identical residue in the five sequences, grey shading means identical residues in at least 80% of the sequences.

FIG. 3: Diagram showing the construction of the functional FadA complex (FadAc) including the multimerization of mFadA via leucine zipper residues and attachment to the inner membrane through the N-terminus of pre-FadA (S. Témoin et al., 2012, FEBS Lett. Vol. 586(1).

FIG. 4: Commassie strained SDS-PAGE and western blot analysis of purified tip-mFadA and cyto-mFadA. Lane were loaded with 1, 2 or 5 μg of purified tip-mFadA or cyto-mFadA.

FIG. 5: A: Map of cosmid vector B: Amino acid sequence of wild-type PE

FIG. 6: A: Dot plot showing expression of PE in 12 colonies transduced with an engineered bacteriophage; the positive control is purified PE.

B: Dot blot showing expression of five C2987 colonies transduced with engineered bacteriophage showing expression of PE; The negative control is untransduced C2987 cells.

C: Western blot demonstrating the PE of the expected size is expressed in C2987 cells transduced with the engineered bacteriophage.

FIG. 7: Graph showing the OD 600 of C2987 which are not transduced with bacteriophage encoding lytic activity (dark green) or with expression of lytic activity after 8 hours and 20 hours (light green) or with expression of lytic activity at 0, 8 and 20 hours (red).

FIG. 8: Graph showing the growth of C2987 as measured by OD600 following induction of lytic activity. The blue line indicated growth after induction of lytic activity at 0 hours; the red line indicates growth after induction of lytic activity after one hour; the grey line indicated growth after indication of lytic activity after two hours.

FIG. 9: Graph showing the expression of GFP as measured by fluorescence in C2987 transduced with bacteriophage encoding both GFP and lytic activity. The blue line indicates GFP expression where lytic activity was induced at 0 hours; the red line indicates GFP expression where lytic activity was induced after 1 hour and the grey line indicates GFP expression where lytic activity was induced after 2 hours.

FIG. 10: Western blot showing expression of PE in C2987 transduced with a bacteriophage encoding PE and lytic activity.

Lane 1 contains 300 ng of PE antigen positive control, Lane 2—induced 0 hr, 50% lysis, Lane 3—induced 0 hr, 100% lysis, Lane 4—induced 1 hr, 50% lysis, Lane 5—induced 1 hr, 100% lysis, Lane 6—induced 2 hr, 50% lysis, Lane 7—induced 2 hr 100% lysis, Lane 8—E. coli cells—negative control

FIG. 11: Presence of specific anti-FadA immune response in faeces 42 days after immunisation with cyto-mFadA. 1, 2 and 3 show ng specific anti-mFadA IgG/μg IgG 42 days after immunisation with 10 μg of adjuvanted cyto-mFadA by the IP route. 4 shows ng specific anti-mFadA IgG/μg IgG 42 days after immunisation with 30 μg of adjuvanted cyto-mFadA by the IG route. 5 shows ng specific anti-mFadA IgG/μg IgG 42 days after immunisation with 60 μg of adjuvanted cyto-mFadA by the IG route.

### DETAILED DESCRIPTION

The present invention discloses a truncated Fusobacterium nucleatum Fusobacterium adhesin A (FadA) protein wherein at least a signal peptide which is at least 80%, 85%, 90% or 95% identical to SEQ ID NO:8 is deleted from the N-terminus of the FadA protein or a truncated Fusobacterium nucleatum Fusobacterium adhesin A (FadA) protein having a truncation at the N-terminus such that at least 14, 15, 16, 17 or 18 of the amino acids at the N-terminus of SEQ ID NO:1 are not present. The truncated FadA protein of the invention is missing at least part of the N-terminal signal sequence resulting in a FadA protein with reduced aggregation in comparison to the full length FadA protein. In am embodiment, the full length FadA protein has the length of SEQ ID NO:1 (129 amino acids long).

As used herein the term “recombinant” means artificial or synthetic.

As used herein the terms “isolated” or “purified” mean a protein, polynucleotide, or vector in a form not found in nature. This includes, for example, a protein, polynucleotide, or vector having been separated from host cell or organism (including crude extracts) or otherwise removed from its natural environment. In certain embodiments, an isolated or purified protein is a protein essentially free from all other polypeptides with which the protein is innately associated (or innately in contact with).

As used herein, the term “subject” refers to an animal, in particular a mammal such as a primate (e.g. human).

As used herein, the term “effective amount,” in the context of administering a therapy (e.g. an immunogenic composition or vaccine of the invention) to a subject refers to the amount of a therapy which has a prophylactic and/or therapeutic effect(s). In certain embodiments, an “effective amount” refers to the amount of a therapy which is sufficient to achieve one, two, three, four, or more of the following effects: (i) reduce or ameliorate the severity of a bacterial infection or symptom associated therewith; (ii) reduce the duration of a bacterial infection or symptom associated therewith; (iii) prevent the progression of a bacterial infection or symptom associated therewith; (iv) cause regression of a bacterial infection or symptom associated therewith; (v) prevent the development or onset of a bacterial infection, or symptom associated therewith; (vi) prevent the recurrence of a bacterial infection or symptom associated therewith; (vii) reduce organ failure associated with a bacterial infection; (viii) reduce hospitalization of a subject having a bacterial infection; (ix) reduce hospitalization length of a subject having a bacterial infection; (x) increase the survival of a subject with a bacterial infection; (xi) eliminate a bacterial infection in a subject; (xii) inhibit or reduce a bacterial replication in a subject; and/or (xiii) enhance or improve the prophylactic or therapeutic effect(s) of another therapy.

FadA Proteins

The present invention provides truncated Fusobacterium nucleatum Fusobacterium adhesin A (FadA) proteins wherein the native FadA polypeptide contains a deletion of amino acid sequence at the N-terminus and/or C-terminus leading to a deletion of at least the first 10, 12, 14, 16 or 18 amino acids (for example 18 amino acids) from the N-terminus of native FadA, such as the FadA of SEQ ID NO:1 or FadA proteins having at least 80%, 85%, 90%, 92% or 95% amino acid sequence identity of SEQ ID NO:1. Therefore at least part of the signal peptide, e.g. the sequence corresponding to first 18 amino acid of SEQ ID NO:1 is deleted from the N-terminus of the FadA protein. It can readily be seen that the initial methionine of the signal sequence can be retained in order to allow expression of the truncated FadA protein (as seen in SEQ ID NO:3 and 4 for example). In an embodiment, such an absent sequence is at least 80%, 85%, 90%, 92%, or 95% identical to SEQ ID NO:8. The truncation results in a FadA protein in which at least part of the signal sequence is missing, resulting in a FadA protein which aggregates less than a full length FadA protein (for example 129 amino acids long).

In an embodiment, the FadA protein is from a particular subspecies of F. nucleatum, for example from subspecies, nucleatum, animals, vincentii, polymorphum or fusiforme. Sequences from F. nucleatum nucleatum are found in SEQ ID NO:1-12. Sequences from F. nucleatum animalis are found in SEQ ID NO:14-17. Sequences from F. nucleatum vincentii are found in SEQ ID NO:18-21. Sequences from F. nucleatum polymorphum are found in SEQ ID NO:22-25.

In an embodiment, the truncated Fusobacterium nucleatum Fusobacterium adhesin A (FadA) protein of the invention has an amino acid sequence which is at least 80%, 85%, 90%, 95%, 97%, 98%, 99% or 100% identical to SEQ ID NO: 2, 3, 4, 5, 6 or 7. These sequences represent FadA proteins with a short (15-25 amino acid) truncation at the N-terminus and other FadA proteins having more extensive truncations of up to 65 amino acids from the N-terminus of a native length FadA, for example up to 20, 30, 40, 50 or 60 amino acids deleted from the N-terminus relative to a native FadA polypeptide such as the FadA of SEQ ID NO:1. In an embodiment up to 21 amino acids are deleted from the C-terminus of a native FadA polypeptide, for example the FadA of SEQ ID NO:1. For example up to 5, 10, 15 or 20 amino acids are deleted from the C-terminus of a native FadA polypeptide, for example the FadA of SEQ ID NO:1. For example, at least 12, at least 14, at least 17 or 12-65, 14-65, 17-65, 18-65, 18-65, 20-60, 30-50 or 50-60 amino acids are deleted from the N-terminus of FadA, for example the FadA of SEQ ID NO:1. For example, 0-25, 0-20, 0-15, 0-10 or 0-5 amino acids are deleted from the C-terminus of native FadA, for example FadA of SEQ ID NO:1.

In an embodiment, at least 18 amino acids are deleted from the N-terminus and at least 5 amino acids are deleted from the C-terminus of native FadA polypeptide. In an embodiment, at least 18 amino acids are deleted from the N-terminus and at least 10 amino acids are deleted from the C-terminus of native FadA polypeptide. In an embodiment, at least 18 amino acids are deleted from the N-terminus and at least 15 amino acids are deleted from the C-terminus of native FadA polypeptide. In an embodiment, at least 18 amino acids are deleted from the N-terminus and at least 20 amino acids are deleted from the C-terminus of native FadA polypeptide. In embodiments where the initial methionine residue is deleted as part of the N-terminal deletion, the methionine can be replaced by a methionine residue or a short peptide containing methionine (such as MAP) in order to allow expression.

In an embodiment, at least 18 amino acids are deleted from the N-terminus and 1-5 amino acids are deleted from the C-terminus of native FadA polypeptide. In an embodiment, at least 18 amino acids are deleted from the N-terminus and 1-10 amino acids are deleted from the C-terminus of native FadA polypeptide. In an embodiment, at least 18 amino acids are deleted from the N-terminus and 1-15 amino acids are deleted from the C-terminus of native FadA polypeptide. In an embodiment, at least 18 amino acids are deleted from the N-terminus and 1-20 amino acids are deleted from the C-terminus of native FadA polypeptide.

In an embodiment, at least 20 amino acids are deleted from the N-terminus and 1-5 amino acids are deleted from the C-terminus of native FadA polypeptide. In an embodiment, at least 20 amino acids are deleted from the N-terminus and 1-10 amino acids are deleted from the C-terminus of native FadA polypeptide. In an embodiment, at least 20 amino acids are deleted from the N-terminus and 1-15 amino acids are deleted from the C-terminus of native FadA polypeptide. In an embodiment, at least 20 amino acids are deleted from the N-terminus and 1-20 amino acids are deleted from the C-terminus of native FadA polypeptide.

In an embodiment, at least 30 amino acids are deleted from the N-terminus and 1-5 amino acids are deleted from the C-terminus of native FadA plypeptide. In an embodiment, at least 30 amino acids are deleted from the N-terminus and 1-10 amino acids are deleted from the C-terminus of native FadA polypeptide. In an embodiment, at least 30 amino acids are deleted from the N-terminus and 1-15 amino acids are deleted from the C-terminus of native FadA polypeptide. In an embodiment, at least 30 amino acids are deleted from the N-terminus and 1-20 amino acids are deleted from the C-terminus of native FadA polypeptide.

In an embodiment, at least 40 amino acids are deleted from the N-terminus and 1-5 amino acids are deleted from the C-terminus of native FadA polypeptide. In an embodiment, at least 40 amino acids are deleted from the N-terminus and 1-10 amino acids are deleted from the C-terminus of native FadA polypeptide. In an embodiment, at least 40 amino acids are deleted from the N-terminus and 1-15 amino acids are deleted from the C-terminus of native FadA polypeptide. In an embodiment, at least 40 amino acids are deleted from the N-terminus and 1-20 amino acids are deleted from the C-terminus of native FadA polypeptide.

In an embodiment, at least 50 amino acids are deleted from the N-terminus and 1-5 amino acids are deleted from the C-terminus of native FadA polypeptide. In an embodiment, at least 50 amino acids are deleted from the N-terminus and 1-10 amino acids are deleted from the C-terminus of native FadA polypeptide. In an embodiment, at least 50 amino acids are deleted from the N-terminus and 1-15 amino acids are deleted from the C-terminus of native FadA polypeptide. In an embodiment, at least 50 amino acids are deleted from the N-terminus and 1-20 amino acids are deleted from the C-terminus of native FadA polypeptide.

In a preferred embodiment, the sequence TRFY (corresponding to amino acids 84-87 in SEQ ID NO:1) is retained in the truncated FadA protein of the invention. In an embodiment the truncated FadA protein comprises SEQ ID NO:5, 26, 27 or 28. In a preferred embodiment the truncated FadA protein comprises an amino acid sequence with an least 80%, 85%, 90%, 95%, 98% 99% or 100% sequence identity to SEQ ID NO:5, 26, 27 or 28.

In an embodiment, the truncated FadA protein of the invention has a sequence helpful for protein purification (a purification tag), for example a histag at the C-terminus of the truncated FadA protein. In further embodiment no purification tag is present in the truncated FadA protein. In an embodiment, a methionine residue is added to the N-terminus of the truncated FadA protein of the invention. In an embodiment, a sequence, “MAP”, is added to the N-terminus of the truncated FadA protein of the invention.

In an embodiment, the truncated FadA protein of the invention is not associated with a further FadA molecule which contains a signal peptide at least 90% identical to MKKFLLLAVL AVSASAFA (SEQ ID NO:8). Thus, the truncated FadA protein of the invention is not in the form of the FadAc complex, described as the active form found in nature, in which a chain of mFadA proteins are attached to a FadA containing a signal peptide attaching the complex to a membrane.

In an embodiment, the truncated FadA protein of the invention comprises a portion having an amino acid sequence at least 80%, 85%, 90%, 95%, 97%, 99% or 100% identity to SEQ ID NO:5. The 44 amino acids of SEQ ID NO:5 contain the TRFY tip sequence and surrounding sequence, optionally to allow the correct folding of the FadA tip. In an embodiment, the truncated FadA protein of the invention comprises at least 40, 44, 45, 50, 55, 60, 70, 80, 90, 100 or 110 amino acids.

In an embodiment, the truncated FadA protein of the invention is capable of generating an immune response against F. nucleatum. In an embodiment, such an immune response is a neutralising immune response, for example a response capable of preventing F. nucleatum from binding to a human cell. Such an immune response is optionally capable blocking the binding of F. nucleatum to E-cadherin.

In an embodiment, the truncated FadA protein of the invention is in an oligomeric form optionally wherein 2-20, 3-15, 4-10, 2-10 or 2-5 truncated FadA proteins are non-covalently associated. In an embodiment, such truncated FadA proteins have an amino acid sequence with at least 90%, 95%, 97%, 98%, 99% or 100% identity to SEQ ID NO:2, 3 or 4.

In an embodiment, the truncated FadA protein of the invention is predominantly in monomeric form, optionally wherein at least 70%, 80%, 90%, 95% or 100% of the truncated FadA protein is in monomeric form. Alternatively, the truncated FadA protein is in dimeric, trimeric or tetrameric form. For example, 2, 3, 4, 5, 6, 7, 8, 9 or 10 truncated FadA proteins of the invention are held together by non-covalent interactions, optionally involving at least 2, 4 or 6 of the leucine residues at positions corresponding to positions 7, 11, 14, 21, 53, 76 and 84 of SEQ ID NO:2. In an embodiment, such a truncated FadA protein has an amino acid sequence which is at least 90%, 95%, 97%, 98%, 99% or 100% identical to SEQ ID NO:5, 6 or 7.

In an embodiment the truncated FadA protein of the invention has at least one point mutation resulting in lower aggregation than a corresponding FadA protein not containing said at least one point mutation. For example, the truncated FadA protein optionally contains a point mutation at a leucine residue selected from the group of residues consisting of leucine 7, 11, 14, 21, 53, 76 and 84 of SEQ ID NO:2 resulting in the substitution of said leucine residue for a different amino acid. In an embodiment, the mutation involves the substitution of a leucine residue with a glycine, valine, methionine, threonine, serine or alanine residue.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see, e.g., Altschul, et al., 1990, J. Mol. Biol. 215: 403-410 and Altschul, et al., 1977, Nucleic Acids Res. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul, et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a wordlength (VV) of 3, an expectation (E) of 10, and the BLOSUM62 scoring matrix (see Henikoff and Henikoff, 1989, Proc Natl Acad Sci USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel, et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided. The ClustalW program is also suitable for determining identity.

In some embodiments, the truncated FadA protein is an isolated polypeptide.

Polynucleotides Encoding FadA Proteins

A further aspect of the invention is a polynucleotide encoding the truncated FadA protein of the invention. In an embodiment, such polynucleotides comprise a nucleotide sequence at least 80%, 85%, 90%, 95%, 97%, 99% or 100% identical to SEQ ID NO:9, 10, 11 or 12.

In an embodiment, the polynucleotide of the invention does not contain the sequence encoding the native signal peptide (SEQ ID NO:8; however, in an embodiment the polynucleotide optionally encodes a heterologous signal sequence at the N-terminus of the recombinant FadA; such a heterologous signal sequence has less than 70%, 60%, 50%, 40%, 30% or 20% identity to SEQ ID NO:8. In an embodiment, such a heterologous signal sequence is capable of targeting the recombinant FadA to the surface of a bacterium. In an embodiment, the heterologous signal sequence is capable of enabling the secretion of the recombinant FadA from a bacterium. In an embodiment, the FadA passes through the membrane and does not become associated with the membrane

A further aspect of the invention is a vector comprising the polynucleotide of the invention wherein the expression of FadA is under the transcriptional control of a promoter. The promoter is optionally a strong promoter which is capable of driving the expression of sufficient FadA for the FadA to be capable of eliciting a strong immune response in a mammalian host. The vector is optionally delivered as part of a viral delivery system, for example a bacteriophage. In this case the vector optionally contains at least one packaging signal to allow the vector to be introduced into a viral delivery system. In an embodiment, the vector contains, 1, 2 or 3 packaging signal sequences. In an embodiment, the packaging signal is a cos sequence. In an embodiment, the vector contains 1, 2, or 3 cos sequences. It is also advantageous for the vector to contain at least one origin of replication. In an embodiment, the vector comprises a bacterial origin of replication. In an embodiment, the vector comprises a bacteriophage origin of replication.

In an embodiment, the vector of the invention is capable of being used as part of a prime and kill bacteriophage described in WO 17/114979. In the prime and kill strategy, a bacteriophage is introduced into a mammalian host with the aim of the bacteriophage infecting bacteria within the mammalian host. The bactreriophage contains polynucleotide encoding a selected antigen under the control of a strong and/or early promoter such that sufficient antigen can be expressed and released from the bacterium in a short time. In an embodiment, the bacteriophage also encodes a killing protein (for example a bacteriophage lysin or a CRISPR-Cas nuclease) so that, following expression of the selected antigen, the bacterium is killed. This approach could be used with FadA as the selected antigen, using a vector of the invention packaged into a bacteriophage targeting F. nucleatum. In this case, a bacteriophage would target F. nucleatum, for example F. nucleatum in the colon or F. nucleatum in the oral cavity. The expression of FadA under the control of a strong promoter would allow the production of sufficient FadA for an immune response to be generated against the FadA (for example by a recombinant bacteriophage being engineered to express the FadA antigen instead of viral coat proteins). The bacteriophage would also kill the F. nucleatum through expression of a protein capable of killing the F. nucleatum, for example a bacteriophage lysin or a CRISPR-Cas nuclease. In this way a proportion of F. nucleatum in the colon would be directly killed by the action of the killing gene in bacteria infected by the bacteriophage. In addition, the release of FadA would allow a second mechanism of targeting further F. nucleatum bacteria through the development of an immune response against FadA by the mammalian host. Since the tip of FadA is an adhesin involved in the attachment of F. nucleatum to mammalian cells, an immune response against FadA has the potential of blocking the binding of FadA to E-cadherin, preventing signal transduction from increasing the replication of cells in the colon.

Therefore, the vector of the invention optionally contains a killing gene which encodes a bacteriophage lysin or a CRISPR-Cas nuclease. In an embodiment, the killing gene is under the control of a weak and/or late promoter so that sufficient FadA can be expressed before the F. nucleatum host is killed. In an embodiment, the expression of FadA is under the control of a strong promoter and the expression of a killing gene is under the control of a strong promoter.

Bacteriophages Encoding FadA Proteins

In a further aspect, the invention provides a recombinant bacteriophage comprising a phage genome polynucleotide including a gene encoding the truncated FadA protein of the invention or a polynucleotide or vector of the invention and the phage genome polynucleotide whether packed into a bacteriophage, for example as part of a delivery system or in isolated form. In an embodiment, the recombinant bacteriophage is capable of infecting a F. nucleatum bacterium. In an embodiment, F. nucleatum is a host bacterium for the recombinant bacteriophage of the invention. In an embodiment, the recombinant bacteriophage is adapted to bind to a host bacterium (e.g. F. nucleatum) and insert the phage genome polynucleotide into said host bacterium (e.g. F. nucleatum). In an embodiment, the recombinant bacteriophage is adapted to bind to a host bacterium (e.g. F. nucleatum) either by the bacteriophage naturally targeting F. nucleatum or through modification of a gene encoding a bacteriophage tail fibre/plate. In an embodiment the host bacterium is a Fusobacterium, for example Fusobacterium nucleatum bacterium, for example Fusobacterium nucleatum nucleatum, Fusobacterium nucleatum animalis, Fusobacterium nucleatum vincentii, Fusobacterium nucleatum polymorphum or Fusobacterium nucleatum fusiforme, suitably Fusobacterium nucleatum nucleatum.

In a further embodiment, the host bacterium is E. coli, optionally a commensal E. coli. In an embodiment, the recombinant bacteriophage is capable of infecting a E. coli bacterium. In an embodiment, E. coli is a host bacterium for the recombinant bacteriophage of the invention. In an embodiment, the recombinant bacteriophage is adapted to bind to a host bacterium (e.g. E. coli) and insert the phage genome polynucleotide into said host bacterium (e.g. E. coli). In an embodiment, the recombinant bacteriophage is adapted to bind to a host bacterium (e.g. E. coli) through modification of a gene encoding a bacteriophage tail fibre/plate. The targeting of E. coli by the bacteriophage of the invention is particular useful for using the E. coli (for example a commensal E. coli) to express the FadA antigen of the invention in the gut. Such expression in the gut allows an immune response against FadA to be raised, optionally in order for the immune response to block the binding of F. nucleatum binding to E-cadherin through FadA. In an embodiment, such a neutralising immune response may alleviate colorectal cancer by preventing F. nucleatum interacting with epithelial cells. In such embodiments it is preferred that the bacteriophage not encode a killing protein such as a lysin or CRISPR-Cas nuclease capable of killing E. coli bacteria.

In a further embodiment, the host bacterium is a streptococcus, optionally an oral commensal Streptococcus selected from the group consisting of S. mitis, S. mutans, S. oralis, S. salivarius and S. sobrinus. In an embodiment, the recombinant bacteriophage is capable of infecting a Streptococcus bacterium. In an embodiment, S. mitis, S. mutans, S. oralis, S. salivarius or S. sobrinus is a host bacterium for the recombinant bacteriophage of the invention. In an embodiment, the recombinant bacteriophage is adapted to bind to a host bacterium (e.g. S. mitis, S. mutans, S. oralis, S. salivarius or S. sobrinus) and insert the phage genome polynucleotide into said host bacterium (e.g. S. mitis, S. mutans, S. oralis, S. salivarius or S. sobrinus). In an embodiment, the recombinant bacteriophage is adapted to bind to a host bacterium (e.g. S. mitis, S. mutans, S. oralis, S. salivarius or S. sobrinus) through modification of a gene encoding a bacteriophage tail fibre/plate.

In an embodiment, the recombinant bacteriophage of the invention comprises the gene encoding the truncated FadA protein under the control of an early promoter or a strong promoter. In an embodiment, the recombinant bacteriophage of the invention comprises a killing gene encoding a protein that is capable of killing a host bacterium, optionally under the control of an early promoter or a strong promoter. In an embodiment, the recombinant bacteriophage of the invention comprises the gene encoding the truncated FadA protein under the control of a late of a weak promoter. In an embodiment, the recombinant bacteriophage of the invention comprises a killing gene encoding a protein that is capable of killing a host bacterium, optionally under the control of a late or a weak promoter

In an embodiment, the recombinant bacteriophage of the invention is selected from the group of families consisting of; myoviridae, siphoviridae, podoviridae, corticiviridae, tectiviridae, leviviridae, cystoviridae, inoviridae, lipothrixviridae, rudiviridae, plasmaviridae and fuselloviridae, for example, the bacteriophage is a myoviridae or a siphoviridae.

In an embodiment, the recombinant bacteriophage of any one of the invention wherein the polynucleotide encoding the truncated FadA contains a heterologous signal sequence which is not the signal sequence found in full length FadA (for example a FadA containing 129 amino acids similar to that encoded by SEQ ID NO:1). For example, the signal sequence is capable of directing the truncated FadA to the surface of a bacterium infected by the bacteriophage. Alternatively, the signal sequence is capable of directing the secretion of the recombinant FadA through the outer membrane of the bacterium infected by the bacteriophage, optionally so that the FadA is released from the bacterium, optionally so that the FadA is mainly not associated with the bacterium. In cases where the FadA protein does not contain a signal sequence, the truncated FadA, after expression is released into the cytoplasm of a bacterium infected by the bacteriophage. In such a case, the truncated FadA is optionally released from the cytoplasm of the bacterium on death of the bacterium.

In an embodiment, the recombinant bacteriophage of the invention is incapable of carrying out a lysogenic cycle or a lytic cycle or both a lysogenic and a lytic cycle. This is optionally achieved by the deletion of genes encoding bacteriophage capsid proteins, bacteriophage lysins and/or genes encoding proteins essential for the lysogenic cycle, optionally resulting in such genes being absent from the bacteriophage genome. In an embodiment, the recombinant bacteriophage is adapted to degrade biofilm.

As used herein “carrying out a lytic cycle” means that the bacteriophage is not only capable of lysing a host cell but also produces progeny bacteriophage which are released as part of a lytic cycle. Therefore a recombinant bacteriophage is incapable of carrying out a lytic cycle if it cannot produce bacteriophage progeny, even if it expresses a bacteriophage lysin or other means of lysing a host cell.

In an further aspect, the invention discloses a recombinant bacteriophage genome polynucleotide comprising a gene encoding the truncated Fusobacterium adhesin A (FadA) of the invention. In an embodiment, the recombinant bacteriophage genome contains a gene encoding a truncated FadA and a killing gene encoding a protein that is capable of killing a host bacterium, for example a bacteriophage lysin or a nuclease associated with CRISPR, optionally Cas9/CRISPR.

In an embodiment, the recombinant bacteriophage genome polynucleotide of the invention comprises at least one bacteriophage packaging sequence, for example a cos sequence. In an embodiment, the recombinant bacteriophage genome polynucleotide comprises a bacteriophage or bacterial origin of replication.

In an embodiment, the recombinant bacteriophage genome polynucleotide contains a gene encoding truncated FadA which is under the control of an early promoter or a strong promoter. In an embodiment, the recombinant bacteriophage genome polynucleotide contains a killing gene under the control of an early promoter or a strong promoter. In an embodiment, the recombinant bacteriophage genome polynucleotide contains a gene encoding truncated FadA which is under the control of a late of a weak promoter. In an embodiment, the recombinant bacteriophage genome polynucleotide contains a killing gene under the control of a late or a weak promoter

In an embodiment, the recombinant bacteriophage genome polynucleotide is from a bacteriophage family selected from the group of families consisting of; myoviridae, siphoviridae, podoviridae, corticiviridae, tectiviridae, leviviridae, cystoviridae, inoviridae, lipothrixviridae, rudiviridae, plasmaviridae and fuselloviridae, for example from a myoviridae or a siphoviridae.

In an embodiment, the recombinant bacteriophage genomic polynucleotide is missing at least one gene associated with a lysogenic cycle which is for example inactivated or deleted. In an embodiment at least one gene encoding a bacteriophage structural protein, for example a capsid protein gene, is deleted. In an embodiment, the recombinant bacteriophage genomic polynucleotide comprises a gene encoding a protein capable of degrading biofilm.

Formulations

The truncated FadA proteins, of the invention are particularly suited for inclusion in immunogenic compositions and vaccines.

The present invention provides an immunogenic composition comprising a truncated FadA protein of the invention, and optionally a pharmaceutically acceptable excipient and/or carrier. The immunogenic composition or vaccine optionally comprises an adjuvant.

Immunogenic compositions comprise an immunologically effective amount of the FadA protein of the invention, as well as any other components. By “immunologically effective amount”, it is meant that the administration of that amount to an individual, either as a single dose or as part of a series is effective for treatment or prevention. This amount varies depending on the health and physical condition of the individual to be treated, age, the degree of protection desired, the formulation of the vaccine and other relevant factors.

Pharmaceutically acceptable excipients and carriers are described, for example, in Remington's Pharmaceutical Sciences, by E. W. Martin, Mack Publishing Co. Easton, PA, 5th Edition (975). Pharmaceutically acceptable excipients can include a buffer, such as a phosphate buffer (e.g. sodium phosphate). Pharmaceutically acceptable excipients can include a salt, for example sodium chloride. Pharmaceutically acceptable excipients can include a solubilizing/stabilizing agent, for example, polysorbate (e.g. TWEEN 80). Pharmaceutically acceptable excipients can include a preservative, for example 2-phenoxyethanol or thiomersal. Pharmaceutically acceptable excipients can include a carrier such as water or saline.

Also provided is a method of making the immunogenic composition of the invention comprising the step of mixing the FadA of the invention with a pharmaceutically acceptable excipient and/or carrier.

The present invention also provides a vaccine comprising an immunogenic composition of the invention and optionally an adjuvant.

The term “adjuvant” refers to a compound that when administered in conjunction with or as part of an immunogenic composition of vaccine of the invention augments, enhances and/or boosts the immune response to FadA, but when the compound is administered alone does not generate an immune response to the modified FadA. Adjuvants can enhance an immune response by several mechanisms including, e.g. lymphocyte recruitment, stimulation of B and/or T cells, and stimulation of macrophages. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see United Kingdom Patent GB2220211), MF59 (Novartis), AS03 (GlaxoSmithKline), AS04 (GlaxoSmithKline), and saponins, such as QS21 (see Kensil et al. in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al. N. Engl. J. Med. 336, 86-91 (1997)).

In certain embodiments, the FadA protein is formulated with an adjuvant comprising an immunologically active saponin fraction presented in the form of a liposome. The adjuvant may further comprise a lipopolysaccharide. The adjuvant may include QS21. For example, in one embodiment, the adjuvant contains QS21 in a liposomal formulation. In one embodiment, the adjuvant system includes 3D-MPL and QS21. For example, in one embodiment, the adjuvant contains 3D-MPL and QS21 in a liposomal formulation. Optionally, the adjuvant system also contains cholesterol. In one specific embodiment, the adjuvant includes QS21 and cholesterol. Optionally, the adjuvant system contains 1,2-Dioleoyl-sn-Glycero-3-phosphocholine (DOPC). For example, in one specific adjuvant system contains cholesterol, DOPC, 3D-MPL and QS21.

In one specific example, the immunogenic composition includes an adjuvant formulated in a dose that includes: from about 0.1 to about 0.5 mg cholesterol; from about 0.25 to about 2 mg DOPC; from about 10 μg to about 100 μg 3D-MPL; and from about 10 μg to about 100 μg QS21. In a further specific example the immunogenic composition includes an adjuvant formulated in a dose that includes: from about 0.1 to about 0.5 mg cholesterol, from about 0.25 to about 2 mg DOPC, from about 10 μg to about 100 μg 3D-MPL, and from about 10 μg to about 100 μg QS21. In one specific formulation, the adjuvant is formulated in a single dose that contains: about 0.25 mg cholesterol; about 1.0 mg DOPC; about 50 μg 3D-MPL; and about 50 μg QS21. In other embodiments, the immunogenic composition is formulated with a fractional dose (that is a dose, which is a fraction of the preceding single dose formulations, such as one half of the preceding quantity of components (cholesterol, DOPC, 3D-MPL and QS21), ¼ of the preceding quantity of components, or another fractional dose (e.g., ⅓, ⅙, etc.) of the preceding quantity of components.

In one embodiment, the immunogenic compositions according to the invention include an adjuvant containing combinations of lipopolysaccharide and Quillaja saponins that have been disclosed previously, for example in EP0671948. This patent demonstrated a strong synergy when a lipopolysaccharide (3D-MPL) was combined with a Quillaja saponin (QS21).

A particularly suitable saponin for use in the present invention is Quil A and its derivatives. Quil A is a saponin preparation isolated from the South American tree Quillaja Saponaria Molina and was first described by Dalsgaard et al. in 1974 (“Saponin adjuvants”, Archiv. für die gesamte Virusforschung, Vol. 44, Springer Verlag, Berlin, p 243-254) to have adjuvant activity. Purified fragments of Quil A have been isolated by HPLC which retain adjuvant activity without the toxicity associated with Quil A (EP 0 362 278), for example QS7 and QS21 (also known as QA7 and QA21). QS21 is a natural saponin derived from the bark of Quillaja saponaria Molina, which induces CD8+ cytotoxic T cells (CTLs), Th1 cells and a predominant IgG2a antibody response and is a preferred saponin in the context of the present invention.

In a specific embodiment, QS21 is provided in its less reactogenic composition where it is quenched with an exogenous sterol, such as cholesterol for example. Several particular forms of less reactogenic compositions wherein QS21 is quenched with an exogenous cholesterol exist. In a specific embodiment, the saponin/sterol is in the form of a liposome structure (WO 96/33739, Example 1). In this embodiment the liposomes suitably contain a neutral lipid, for example phosphatidylcholine, which is suitably non-crystalline at room temperature, for example eggyolk phosphatidylcholine, dioleoyl phosphatidylcholine (DOPC) or dilauryl phosphatidylcholine. The liposomes may also contain a charged lipid which increases the stability of the lipsome-QS21 structure for liposomes composed of saturated lipids. In these cases the amount of charged lipid is suitably 1-20% w/w, preferably 5-10%. The ratio of sterol to phospholipid is 1-50% (mol/mol), suitably 20-25%.

Suitable sterols include β-sitosterol, stigmasterol, ergosterol, ergocalciferol and cholesterol. In one particular embodiment, the adjuvant composition comprises cholesterol as sterol. These sterols are well known in the art, for example cholesterol is disclosed in the Merck Index, 11th Edn., page 341, as a naturally occurring sterol found in animal fat.

Where the active saponin fraction is QS21, the ratio of QS21:sterol will typically be in the order of 1:100 to 1:1 (w/w), suitably between 1:10 to 1:1 (w/w), and preferably 1:5 to 1:1 (w/w). Suitably excess sterol is present, the ratio of QS21:sterol being at least 1:2 (w/w). In one embodiment, the ratio of QS21:sterol is 1:5 (w/w). The sterol is suitably cholesterol.

In one embodiment, the invention provides a dose of an immunogenic composition comprising immunologically active saponin, preferably QS21, at a level of 60 μg or less, for example between 1 and 60 μg. In one embodiment, the dose of the immunogenic composition comprises QS21 at a level of approximately around 50 μg, for example between 45 and 55 μg, suitably between 46-54 μg or between 47 and 53 μg or between 48 and 52 μg or between 49 and 51 μg, or 50 μg per dose.

In another embodiment the dose of the immunogenic composition comprises QS21 at a level of around 25 μg, for example between 20-30 μg, suitably between 21-29 μg or between 22 and 28 μg or between 23 and 27 μg or between 24 and 26 μg, or 25 μg.

In another embodiment, the dose of the immunogenic composition comprises QS21 at a level of around 10 μg per, for example between 5 and 15 μg, suitably between 6 and 14 μg, for example between 7 and 13 μg or between 8 and 12 μg or between 9 and 11 μg, or 10 μg.

Specifically, a 0.5 ml vaccine dose volume contains 25 μg or 50 μg of QS21 per dose. Specifically, a 0.5 ml vaccine dose volume contains 50 μg of QS21 per dose.

The lipopolysaccharide may be a non-toxic derivative of lipid A, particularly monophosphoryl lipid A or more particularly 3-Deacylated monophoshoryl lipid A (3D-MPL).

3D-MPL is sold under the name MPL by GlaxoSmithKline Biologicals N.A. and is referred throughout the document as MPL or 3D-MPL. See, for example, U.S. Pat. Nos. 4,436,727; 4,877,611; 4,866,034 and 4,912,094. 3D-MPL primarily promotes CD4+ T cell responses with an IFN-γ (Th1) phenotype. 3D-MPL can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3D-MPL is used. Small particle 3D-MPL has a particle size such that it may be sterile-filtered through a 0.22 μm filter. Such preparations are described in WO 94/21292.

The invention therefore provides a dose of an immunogenic composition comprising lipopolysaccharide, preferably 3D-MPL, at a level of 75 μg or less, for example between 1 and 60 μg. In one embodiment the lipopolysaccharide is present at an amount of about 50 μg per dose.

In one embodiment, the dose of the immunogenic composition comprises 3D-MPL at a level of around 50 μg, for example between 45-55 μg, suitably between 46-54 μg or between 47 and 53 μg or between 48 and 52 μg or between 49 and 51 μg, or 50 μg.

In one embodiment, the dose of the immunogenic composition comprises 3D-MPL at a level of around 25 μg, for example between 20-30 μg, suitably between 21-29 μg or between 22 and 28 μg or between 23 and 27 μg or between 24 and 26 μg, or 25 μg.

In another embodiment, the dose of the immunogenic composition comprises 3D-MPL at a level of around 10 μg, for example between 5 and 15 μg, suitably between 6 and 14 μg, for example between 7 and 13 μg or between 8 and 12 μg or between 9 and 11 μg, or 10 μg.

In one embodiment, the volume of the dose is 0.5 ml. In a further embodiment, the immunogenic composition is in a volume suitable for a dose which volume is higher than 0.5 ml, for example 0.6, 0.7, 0.8, 0.9 or 1 ml. In a further embodiment, the human dose is between 1 ml and 1.5 ml.

Specifically, a 0.5 ml vaccine dose volume contains 25 μg or 50 μg of 3D-MPL per dose. Specifically, a 0.5 ml vaccine dose volume contains 50 μg of 3D-MPL per dose.

The dose of the immunogenic composition according to any aspect of the invention suitably refers to human dose. By the term “human dose” is meant a dose which is in a volume suitable for human use. Generally this is between 0.3 and 1.5 ml. In one embodiment, a human dose is 0.5 ml. In a further embodiment, a human dose is higher than 0.5 ml, for example 0.6, 0.7, 0.8, 0.9 or 1 ml. In a further embodiment, a human dose is between 1 ml and 1.5 ml.

Suitable compositions of the invention are those wherein liposomes are initially prepared without MPL (as described in WO 96/33739), and MPL is then added, suitably as small particles of below 100 nm particles or particles that are susceptible to sterile filtration through a 0.22 μm membrane. The MPL is therefore not contained within the vesicle membrane (known as MPL out). Compositions where the MPL is contained within the vesicle membrane (known as MPL in) also form an aspect of the invention.

In a specific embodiment, QS21 and 3D-MPL are present in the same final concentration per dose of the immunogenic composition. In one aspect of this embodiment, a dose of immunogenic composition comprises a final level of 25 μg of 3D-MPL and 25 μg of QS21 or 50 μg of 3D-MPL and 50 μg of QS21.

In a further embodiment, the vaccine comprises an oil-in-water emulsion adjuvant. The oil in water emulsion comprises a metabolisable oil and an emulsifier and optionally a tocol. The metabolisable oil may be present at an amount of about 5.35 mg. The tocol may be present at an amount of about 5.94 mg. Suitably, the emulsifying agent may be present at an amount of about 2.425 mg. Suitably, the metabolisable oil is squalene, the tocol is alpha-tocopherol and the emulsifying agent is polyoxyethylene sorbitan monooleate.

For example, the oil-in-water emulsion can include an oil phase that incorporates a metabolisable oil, and an additional oil phase component, such as a tocol. The oil-in-water emulsion may also contain an aqueous component, such as a buffered saline solution (e.g., phosphate buffered saline). In addition, the oil-in-water emulsion typically contains an emulsifier. In one embodiment, the metabolizable oil is squalene. In one embodiment, the tocol is alpha-tocopherol. In one embodiment, the emulsifier is a nonionic surfactant emulsifier (such as polyoxyethethylene sorbitan monooleate, TWEEN80™). In exemplary embodiments, the oil-in-water emulsion contains squalene and alpha tocopherol in a ratio which is equal or less than 1 (w/w).

The metabolisable oil in the oil-in-water emulsion may be present in an amount of 0.5-10 mg. The tocol in the oil-in-water emulsion may be present in an amount of 0.5-11 mg. The emulsifying agent may be present in an amount of 0.4-4 mg,

In order for any oil in water composition to be suitable for human administration, the oil phase of the emulsion system has to comprise a metabolisable oil. The meaning of the term metabolisable oil is well known in the art. Metabolisable can be defined as ‘being capable of being transformed by metabolism’ (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts, seeds, and grains are common sources of vegetable oils. Synthetic oils are also part of this invention and can include commercially available oils such as NEOBEE® (caprylic/capric triglycerides made using glycerol from vegetable oil sources and medium-chain fatty acids (MCTs) from coconut or palm kernel oils) and others. A particularly suitable metabolisable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolisable oil by virtue of the fact that it is an intermediate in the biosynthesis of cholesterol (Merck index, 10th Edition, entry no. 8619).

Suitably the metabolisable oil is present in the adjuvant composition in an amount of 0.5-10 mg, preferably 1-10, 2-10, 3-9, 4-8, 5-7, or 5-6 mg (e.g. 2-3, 5-6, or 9-10 mg), specifically about 5.35 mg.

Tocols are well known in the art and are described in EP0382271. Suitably the tocol is alpha-tocopherol or a derivative thereof such as alpha-tocopherol succinate (also known as vitamin E succinate). Said tocol is suitably present in in an amount of 0.5-11 mg, preferably 1-11, 2-10, 3-9, 4-8, 5-7, 5-6 mg (e.g. 10-11, 5-6, 2.5-3.5 or 1-3 mg). In a specific embodiment the tocol is present in an amount of about 5.94 mg.

The oil in water emulsion further comprises an emulsifying agent. The emulsifying agent may suitably be polyoxyethylene sorbitan monooleate. In a particular embodiment the emulsifying agent may be Polysorbate® 80 (Polyoxyethylene (20) sorbitan monooleate) or Tween® 80.

Said emulsifying agent is suitably present in the adjuvant composition in an amount of 0.1-5, 0.2-5, 0.3-4, 0.4-3 or 2-3 mg (e.g. 0.4-1.2, 2-3 or 4-5 mg) emulsifying agent. In a specific embodiment the emulsifying agent is present in an amount of about 0.97 mg or about 2.425 mg.

In a further embodiment, flagellin acts as an adjuvant. In an embodiment, flagellin is encoded in a vector or recombinant bacteriophage genome as part of a fusion protein containing FadA of the invention and flagellin. In an embodiment, the truncated FadA protein is part of a fusion protein comprising flagellin.

Also provided is a method of making the immunogenic composition of the invention comprising the step of mixing the FadA protein of the invention with a pharmaceutically acceptable excipient and/or carrier and an adjuvant. Vaccine preparation is generally described in Vaccine Design (“The subunit and adjuvant approach” (eds Powell M. F. & Newman M. J.) (1995) Plenum Press New York).

The immunogenic compositions of the invention can be included in a container, pack, or dispenser together with instructions for administration.

The immunogenic compositions or vaccines of the invention can be stored before use, e.g. 30 the compositions can be stored frozen (e.g. at about −20° C. or at about −70° C.); stored in refrigerated conditions (e.g. at about 4° C.); or stored at room temperature. The immunogenic compositions or vaccines of the invention may be stored in solution or lyophilized. In an embodiment, the solution is lyophilized in the presence of a sugar such as sucrose, trehalose or lactose. In another embodiment, the vaccines of the invention are lyophilized 35 and extemporaneously reconstituted prior to use.

Administration and Dosage

Immunogenic compositions or vaccines of the invention may be used to protect or treat a subject (e.g. mammal, optionally a human), by means of administering said immunogenic composition or vaccine via systemic or mucosal route. These administrations may include injection via the intramuscular (IM), intraperitoneal (IP), intradermal (ID), intranasal (IN) or subcutaneous (SC) routes; or via mucosal administration to the oral/alimentary, rectal, respiratory, genitourinary tracts. Administration via the oral, nasal or rectal route is preferred. Administration via the oral or rectal route is preferred for the treatment or prevention of colorectal cancer. Administration via the oral or nasal route is preferred for the treatment or prevention of periodontitis. In an embodiment for rectal delivery, administration is using a suppository. Administration via the oral route for the treatment or prevention of colorectal cancer is optionally using a tablet. In an embodiment, the tablet is formulated to allow release of the truncated FadA protein or the vector or the bacteriophage of the invention in the alimentary canal, for example in the colon. Adminstration via the oral route for the treatment or prevention of periodontitis is optionally using a mouthwash or a wafer or tablet formulated to dissolve in the mouth. In an embodiment, oral delivery of FadA uses flaggelin as an adjuvant, optionally in the form of a FadA-flagellin fusion protein.

In one aspect, the immunogenic composition or vaccine of the invention is administered by the intramuscular delivery route. Intramuscular administration may be to the thigh or the upper arm. Injection is typically via a needle (e.g. a hypodermic needle), but needle-free injection may alternatively be used. A typical intramuscular dose is 0.5 ml.

The amount of FadA in each immunogenic composition or vaccine dose is selected as an amount which induces an immunoprotective response without significant, adverse side effects in typical vaccines. Such amount will vary depending upon which specific immunogen is employed and how it is presented. The content of FadA protein will typically be in the range 1-100 μg, suitably 5-50 μg. Where FadA is delivered in as a bacteriophage, it is typically delivered at a MOI of 10-10,000 bacteriophage per Fusobacterium.

Prophylactic and Therapeutic Uses

The present invention provides a method of inducing an immune response in a subject (e.g. human), the method comprising administering a immunologically effective amount of the truncated FadA protein, polynucleotide or vector of bacteriophage of the invention, an immunogenic composition of the invention or a vaccine of the invention, to a subject (e.g. human) in need thereof. The present invention also provides a truncated FadA protein, polynucleotide, vector or bacteriophage of the invention, an immunogenic composition of the invention or a vaccine of the invention, for use in inducing an immune response in a subject (e.g. human). The present invention also provides a truncated FadA protein, polynucleotide, vector or bacteriophage of the invention, the immunogenic composition of the invention or the vaccine of the invention in the manufacture of a medicament for inducing an immune response in a subject (e.g. human).

Also provided herein are methods of inducing an immune response in a subject against a bacterium (e.g. Fusobacterium nucleatum), comprising administering to the subject a FadA of the invention an immunogenic composition of the invention or a vaccine of the invention.

The FadA of the invention an immunogenic composition of the invention or a vaccine of the invention can be used to induce an immune response against a bacterium, e.g. Fusobacterium nucleatum In an embodiment, the FadA of the invention an immunogenic composition of the invention or a vaccine of the invention can be used to induce an immune response against a bacterium, e.g. Fusobacterium nucleatum. In one embodiment, said subject has bacterial infection at the time of administration. In another embodiment, said subject does not have a bacterial infection at the time of administration.

Also provided herein are methods of inducing the production of opsonophagocytic antibodies in a subject against a bacterium, comprising administering to the subject a FadA of the invention an immunogenic composition of the invention or a vaccine of the invention. The FadA of the invention an immunogenic composition of the invention or a vaccine of the invention can be used to induce the production of opsonophagocytic antibodies in a subject against a bacterium, e.g. Fusobacterium nucleatum. In an embodiment, the FadA of the invention an immunogenic composition of the invention or a vaccine of the invention can be used to induce the production of opsonophagocytic antibodies in a subject against a bacterium, e.g. Fusobacterium nucleatum.

The present invention also provides methods of treating and/or preventing a bacterial infection in a subject comprising administering to the subject a FadA (truncated protein, polynucleotide, vector or bacteriophage) of the invention. The FadA may be in the form of an immunogenic composition or vaccine. Thus the present invention provides a method of treating and/or preventing a bacterial infection (e.g. Fusobacterium nucleatum) in a subject (e.g. human), the method comprising administering an immunologically effective amount of a FadA of the invention, an immunogenic composition of the invention or a vaccine of the invention, to a subject (e.g. human) in need thereof. The present invention also provides a FadA of the invention, an immunogenic composition of the invention or a vaccine of the invention, for use in treating and/or preventing a bacterial (e.g. Fusobacterium nucleatum) infection in a subject (e.g. human). The present invention also provides a FadA of the invention, the immunogenic composition of the invention or the vaccine of the invention in the manufacture of a medicament for treating and/or preventing a bacterial infection (e.g. Fusobacterium nucleatum) in a subject (e.g. human). In a specific embodiment, the immunogenic composition or vaccine of the invention is used in the prevention of infection of a subject by a Fusobacterium nucleatum nucleatum.

Methods of Treatment of Colorectal Cancer

The present invention further provides a method of treatment or prevention of colorectal cancer comprising the step of: a) administering the truncated FadA protein, or the vector, or the recombinant bacteriophage, or the recombinant bacteriophage genomic polynucleotide of the invention, to a patient in need thereof, optionally a human patient. The method of treatment optionally comprises the further steps of b) entry of the bacteriophage genome polynucleotide into a Fusobacterium nucleatum, and c) expression of the truncated FadA at a sufficient level for an immune response to be elicited against the heterologous protein. The method of treatment of colorectal cancer optionally comprises a further step of d) expression of a killing gene leading to the killing of a host bacterium, for example a F. nucleatum bacterium.

Analogous uses and methods of manufacture are also contemplated. For the treatment of prevention of colorectal cancer, administration of FadA is optionally carried out via oral or rectal routes. The FadA is optionally administered as a polypeptide or a vector, or a phage genome polynucleotide or a bacteriophage encoding FadA which is administered as a suppository or as a tablet.

Methods of Treating Periodontitis

The present invention further provides a method of treatment or prevention of peridontitis comprising the step of: a) administering the truncated FadA protein, or the vector, or the recombinant bacteriophage, or the recombinant bacteriophage genomic polynucleotide of the invention, to a patient in need thereof, optionally a human patient. The method of treatment optionally comprises the further steps of b) entry of the phage genome polynucleotide into a Fusobacterium nucleatum, and c) expression of the recombinant FadA at a sufficient level for an immune response to be elicited against the heterologous protein. The method of treatment of periodontitis optionally comprises a further step of d) expression of a killing gene leading to the killing of a host bacterium, for example a F. nucleatum bacterium.

Analogous uses and methods of manufacture are also contemplated. For the treatment of prevention of peridontitis, administration of FadA is optionally carried out via oral route or the nasal route. The FadA is optionally administered as a polypeptide or a vector, or a phage genome polynucleotide or a bacteriophage encoding FadA which is administered as a mouthwash or a tablet or wafer formulated such that it dissolves in the mouth.

The invention is further disclosed in the following paragraphs:
1. A truncated Fusobacterium nucleatum Fusobacterium adhesin A (FadA) protein wherein at least a signal peptide which is at least 80%, 85%, 90% or 95% identical to SEQ ID NO:8 is deleted from the N-terminus of the FadA protein; or a truncated Fusobacterium nucleatum Fusobacterium adhesin A (FadA) protein having a truncation at the N-terminus such that at least 14, 15, 16, 17 or 18 of the amino acids at the N-terminus of SEQ ID NO:1 are not present.
2. The truncated Fusobacterium nucleatum Fusobacterium adhesin A (FadA) protein of paragraph 1 having an amino acid sequence which is at least 80%, 85%, 90%, 95%, 97%, 98%, 99% or 100% identical to SEQ ID NO: 2-7 or 14-25.
3. The truncated FadA protein of paragraph 1 or 2 which is not associated with a further FadA molecule which contains a signal peptide at least 90% identical to MKKFLLLAVL AVSASAFA (SEQ ID NO:8).
4. The truncated FadA protein of any one of paragraphs 1-3 comprising a portion having an amino acid sequence at least 80% identity to SEQ ID NO:5, 26, 27 or 28.
5. The truncated FadA protein of any one of paragraphs 1-4 comprising at least 40, 45, 50, 55, 60, 70, 80, 90, 100 or 110 amino acids.
6. The truncated FadA protein of any one of paragraphs 1-5 which is capable of generating an immune response against F. nucleatum.
7. The truncated FadA protein of any one of paragraphs 1-6 which is capable of eliciting an immune response which is capable of preventing F. nucleatum from binding to a human cell.
8. The truncated FadA protein of any one of paragraphs 1-7 which is capable of eliciting an immune response which blocks the binding of F. nucleatum to E-cadherin.
9. The truncated FadA protein of any one of paragraphs 1-8 which is in an oligomeric form optionally wherein 2-20, 3-15, 4-10, 2-10 or 2-5 truncated FadA proteins are non-covalently bound together.
10. The truncated FadA protein of paragraph 9 having an amino acid sequence with at least 90% identity to SEQ ID NO:2, 3 or 4.
11. The truncated FadA protein of any one of paragraphs 1-8 which is predominantly in monomeric form, optionally wherein at least 70%, 80%, 90%, 95% or 100% of the truncated FadA protein is in monomeric form.
12. The truncated FadA of paragraph 11 having an amino acid sequence which is at least 90% identical to SEQ ID NO:5, 6 or 7.
13. The truncated FadA protein of any one of paragraphs 1-12 having at least one point mutation resulting in lower aggregation than a corresponding FadA protein not containing said at least one point mutation.
14. The truncated FadA protein of any one of paragraphs 1-13 containing a point mutation at a leucine residue selected from the group of residues consisting of leucine 7, 11, 14, 21, 53, 76 and 84 of SEQ ID NO:2 resulting in the substitution of said leucine residue with a different amino acid.
15. The truncated FadA protein of paragraph 14 wherein the different amino acid is a glycine, valine, methionine, threonine, serine or alanine residue.
16. A polynucleotide encoding the truncated FadA of any one of paragraphs 1-15.
17. The polynucleotide of paragraph 16 having a nucleotide sequence at least 80%, 85%, 90%, 95%, 97%, 99% or 100% identical to SEQ ID NO:9, 10, 11 or 12.
18. The polynucleotide of paragraph 16 or 17 encoding a signal sequence at the N-terminus of the FadA which has less than 70%, 60%, 50% or 40% identity to SEQ ID NO:8.
19. The polynucleotide of paragraph 18 wherein the signal sequence is capable of targeting the FadA to the surface of a bacterium.
20. The polynucleotide of paragraph 18 wherein the signal sequence is capable of enabling the secretion of the FadA from a bacterium.
21. A vector comprising the polynucleotide of any one of paragraphs 16-20 wherein the expression of FadA is under transcriptional control of a promoter.
22. The vector of paragraph 21 wherein the expression of FadA is under the transcriptional control of a strong promoter.
23. The vector of paragraph 21 or 21 further comprising at least one packaging signal.
24. The vector of paragraph 23 wherein the at least one packaging signal is a cos sequence.
25. The vector of paragraph 23 or 24 comprising at least 2 or 3 packaging signals.
26. The vector of any one of paragraphs 21-25 comprising a bacterial origin of replication.
27. The vector of any one of paragraphs 21-26 comprising a bacteriophage origin of replication.
28. The vector of any one of paragraphs 21-27 further comprising a killing gene.
29. The vector of paragraph 28 wherein the killing gene encodes a bacteriophage lysin.
30. The vector of paragraph 28 wherein the killing gene encodes a CRISPR-cas nuclease.
31. The vector of any one of paragraphs 28-30 wherein the killing gene is under the control of a weak and/or late promoter.
32. A recombinant bacteriophage comprising a phage genome polynucleotide including a gene encoding the truncated FadA protein of any one of paragraphs 1-15 or the polynucleotide of any one of paragraphs 16-20.
33. The recombinant bacteriophage of paragraph 32 wherein the recombinant bacteriophage is adapted to bind to a host bacterium and insert the phage genome 30 polynucleotide into said host bacterium.
34. The recombinant bacteriophage of paragraph 32 or 33 wherein the recombinant bacteriophage is adapted to bind to a host bacterium through modification of a gene encoding a bacteriophage tail fibre/plate.
35. The recombinant bacteriophage of any one of paragraphs 32-34 wherein the host bacterium is a Fusobacterium, optionally a Fusobacterium nucleatum bacterium or E. coli or Streptococcus.
36. The recombinant bacteriophage of any one of paragraphs 32-35 wherein the gene encoding the recombinant FadA protein is under the control of an early promoter or a strong promoter.
37. The recombinant bacteriophage of any one of paragraphs 32-36, wherein the phage genome polynucleotide comprises a killing gene encoding a protein that is capable of killing a host bacterium.
38. The recombinant bacteriophage of paragraph 37 wherein the killing gene is under the control of a late or a weak promoter.
39. The recombinant bacteriophage of any one of paragraphs 32-38, wherein the bacteriophage is selected from the group of families consisting of; myoviridae, siphoviridae, podoviridae, corticiviridae, tectiviridae, leviviridae, cystoviridae, inoviridae, lipothrixviridae, rudiviridae, plasmaviridae and fuselloviridae.
40. The recombinant bacteriophage of paragraph 39, wherein the bacteriophage is a myoviridae or a siphoviridae.
41. The recombinant bacteriophage of any one of paragraphs 32-40 wherein the polynucleotide encoding the truncated FadA contains a heterologous signal sequence.
42. The recombinant bacteriophage of paragraph 41 wherein the signal sequence is capable of directing the truncated FadA to the surface of a bacterium infected by the bacteriophage.
43. The recombinant bacteriophage of paragraph 41 wherein the signal sequence is capable of directing the secretion of the truncated FadA through the outer membrane of the bacterium infected by the bacteriophage.
44. The recombinant bacteriophage of any one of paragraph 32-40 wherein the truncated FadA, after expression is released into the cytoplasm of a bacterium 35 infected by the bacteriophage.
45. The recombinant bacteriophage of paragraph 44 wherein the truncated FadA is released from the cytoplasm of the bacterium on death of the bacterium.
46. The recombinant bacteriophage of any one of paragraphs 32-45, wherein the recombinant bacteriophage is incapable of carrying out a lysogenic cycle and/or a lytic cycle.
47. The recombinant bacteriophage of any one of paragraph 32-46, wherein the recombinant bacteriophage is adapted to degrade biofilm.
48. A recombinant bacteriophage genome polynucleotide comprising a gene encoding truncated Fusobacterium adhesin A (FadA) according to any one of paragraphs 1-15 or the polynucleotide of any one of paragraphs 16-20.
49. The recombinant bacteriophage genome of paragraph 48 wherein the gene encodes truncated FadA and a killing gene encoding a protein that is capable of killing a host bacterium.
50. The recombinant bacteriophage genome of paragraph 49 wherein the killing gene encodes a bacteriophage lysin.
51. The recombinant bacteriophage genome of paragraph 49 wherein the killing gene encodes an encodes a CRISPR-Cas (clustered regularly interspaced short palindromic repeat (CRISPR)-CRISPR-associated protein (Cas)) which is optionally capable of targeting gene(s) important for the viability or the pathogencity of the bacteria.
52. The recombinant bacteriophage genome polynucleotide of any one of paragraphs 48-51 comprising at least one bacteriophage packaging sequence.
53. The recombinant bacteriophage genome polynucleotide of any one of paragraphs 48-52 comprising a bacteriophage or bacterial origin of replication.
54. The recombinant bacteriophage genome polynucleotide of any one of paragraphs 48-53 wherein the gene encoding truncated FadA is under the control of an early 30 promoter or a strong promoter.
55. The recombinant bacteriophage genome polynucleotide of any one of paragraphs 48-54 wherein the killing gene is under the control of a late or a weak promoter or an early or strong promoter.
56. The recombinant bacteriophage genome polynucleotide of any one of paragraphs 48-55, wherein the bacteriophage is selected from the group of families consisting of; myoviridae, siphoviridae, podoviridae, corticiviridae, tectiviridae, leviviridae, cystoviridae, inoviridae, lipothrixviridae, rudiviridae, plasmaviridae and fuselloviridae.
57. The recombinant bacteriophage genome polynucleotide of paragraph 56, wherein the bacteriophage is a myoviridae or a siphoviridae.
58. The recombinant bacteriophage genomic polynucleotide of any one of paragraphs 48-57, wherein at least one gene associated with a lysogenic cycle is inactivated.
59. The recombinant bacteriophage genomic polynucleotide of any one of paragraphs 48-58, wherein at least one gene encoding a bacteriophage structural protein is deleted.
60. The recombinant bacteriophage genomic polynucleotide of any one of paragraphs 48-59, comprising a gene encoding a protein capable of degrading biofilm.
61. A pharmaceutical composition comprising the truncated FadA protein of any one of paragraphs 1-15, the polynucleotide of any one of paragraphs 16-20, the vectors of any one of paragraphs 21-31, the recombinant bacteriophage of any one of paragraphs 32-47 or the recombinant bacteriophage genome polynucleotide of any one of paragraphs 48-60.
62. The pharmaceutical composition of paragraph 61, formulated as a tablet, capsule, suppository or mouthwash.
63. A vaccine comprising the recombinant FadA of any one of paragraphs 1-15, the polynucleotide of any one of paragraphs 16-20, the vector of any one of paragraphs 21-31, the recombinant bacteriophage of any one of paragraphs 32-47 or the recombinant bacteriophage genome polynucleotide of any one of paragraphs 48-60, 25 optionally containing an adjuvant.
64. The vaccine of paragraph 63 comprising an adjuvant containing a lipopolysaccharide (optionally 3D-MPL) or an oil-in-water emulsion.
65. The vaccine of paragraph 63 comprising an adjuvant which is flagellin.
66. The vaccine of paragraph 65 wherein the flagellin is encoded in the vector or the recombinant bacteriophage genome polynucleotide.
67. A recombinant FadA of any one of paragraphs 1-15, the polynucleotide of any one of paragraphs 16-20, the vector of any one of paragraphs 21-31, the recombinant bacteriophage of any one of paragraphs 32-47 or the recombinant bacteriophage genome polynucleotide of any one of paragraphs 48-60 for use in therapy.
68. The pharmaceutical composition of paragraph 61 or 62 or the vaccine of any one of paragraphs 63-66 for use in the treatment or prevention of colorectal cancer in a human.
69. A method of treatment of colorectal cancer comprising the step of: a) administering the truncated FadA protein of any one of paragraphs 1-15 or the vector of any one of paragraphs 21-31 or the recombinant bacteriophage of any one of paragraphs 32-47 or the recombinant bacteriophage genomic polynucleotide according to any one of paragraphs 48-60 or the vaccine of any one of paragraphs 63-66, to a patient in need thereof, optionally a human patient.
70. A method of treatment of peridontitis comprising the step of: a) administering the truncated FadA protein of any one of paragraphs 1-15 or the vector of any one of paragraphs 21-31 or the recombinant bacteriophage of any one of paragraphs 32-47 or the recombinant bacteriophage genomic polynucleotide according to any one of paragraphs 48-60 or the vaccine of any one of paragraphs 63-66, to a patient in need thereof, optionally a human patient.
71. The method of treatment of paragraph 69 or 70 where dependent on any one of paragraphs 32-60 comprising the further steps of b) entry of the phage genome polynucleotide into a Fusobacterium nucleatum, and c) expression of the truncated FadA protein at a sufficient level for an immune response to be elicited against the heterologous protein.
72. The method of treatment of paragraph 71, wherein the method comprises a further step of d) expression of a killing gene leading to the killing of the Fusobacterium nucleatum.
73. The method of treatment of any one of claims 69-72 wherein oral administration is used.
74. The method of treatment of claim 73 wherein a tablet is used, optionally a tablet 30 which disperses in the alimentary canal, optionally in the colon, optionally in the mouth.

In order that this invention may be better understood, the following examples are set forth. These examples are for purposes of illustration only, and are not to be construed as limiting the scope of the invention in any manner.

### EXAMPLES

### Example 1—Design of FadA Antigens

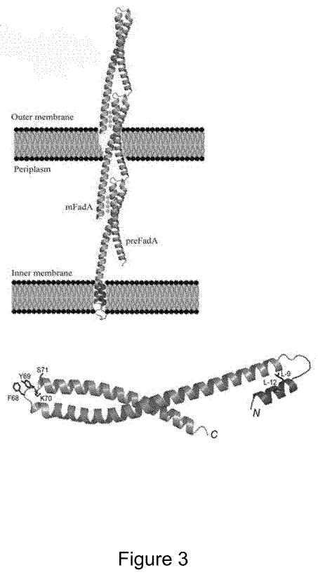

FadA is a novel small and highly conserved (among fusobacteria species) adhesin of Fusobacterium nucleatum, having 129 amino acids in its full length form. There are two forms of FadA; i) pre-FadA (non secreted form, containing a signal peptide) and mature FadA (mFadA, secreted), which together constitute the functional FadA complex (FadAc). FIG. 3 illustrates how FadAc is thought to be assembled. This protein is involved in bacterial attachment and invasion to host cells. A FadA-deleted mutant is defective in host-cell attachment and invasion as well as colonization in murine placenta. However, a complementation with FadA restores host-cell adhesion, invasion and colonization of the placenta.

An inhibitory cell attachment assay shows that the pre-FadA-mFadA complex (FadAc) inhibited the attachment of F. nucleatum to host cells, while mFadA alone did not (Temoin et al FEBS Lett. 586; 1-6 (2012)). According to the teaching of Temoin et al, the full length FadA which is capable of assembling into the FadAc active complex should be considered as a candidate for use in immunisation.

A proposed model of FadA filament formation is shown in FIG. 3: i) mFadA is secreted across inner membrane and self assembles in the periplasm; ii) as more mFadA polypeptides are synthesized and secreted, this leads to the elongation of the FadA chain which extends across the outer membrane; iii) when elongation of the filament stops, pre-FadA is incorporated to the base. It is envisaged that the N-terminal part of pre-FadA forms a short helix connected to the mFadA helix via a hairpin to form interhelical contacts potentially mediated via Leu residues on both helices. The N-terminal helical hairpin could serve as an anchor in the inner membrane.

Several FadA antigens were designed to establish vaccine candidates with the properties of i) containing the tip section of FadA which is thought to be involved in binding to cells and ii) achieving a soluble form of FadA.

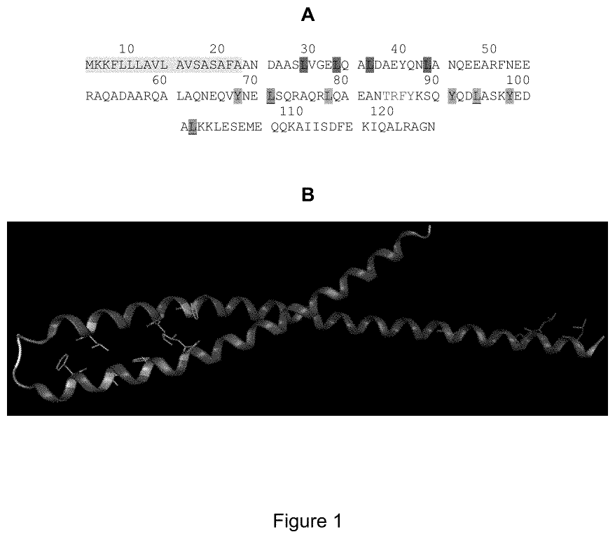

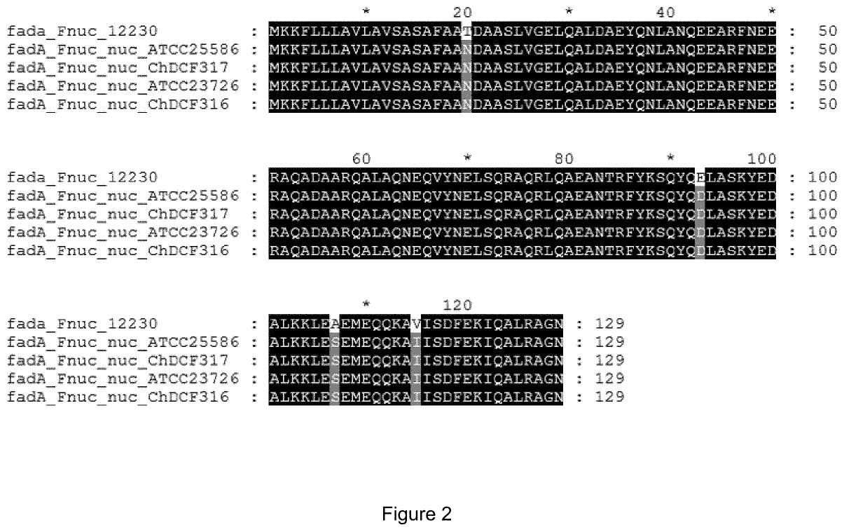

The sequence alignment shown in FIG. 2 shows a high degree of identity between FadA amino acid sequence from different strains. The reference sequence chosen for our prototypes was F. nucleatum subsp. nucleatum strain ATCC25586. This FadA sequence is identical to that found in other strains as shown in FIG. 2. However, it is envisaged that FadA from other subspecies could be used in a vaccine.

The following sequence is an annotated FadA ATCC25586 sequence (based on annotation of F. nucleatum subspecies polymorphum strain 12230):

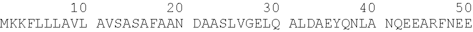

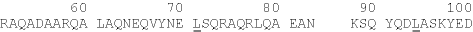

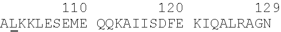

The N-terminus of the sequence contain an 18 amino acid long signal peptide (yellow highlight). Two coiled-coils extend between amino acids 22-81 and amino acids 88-122. These are separated by a central haripin or tip including the red amino acids TRFY (amino acids 84-87).

Several leucine (Leu or L) amino acids are important for either stablising the structure of FadA through intramolecular interactions with tyrosine residues, whereas other leucine stabilise interhelical interactions with adjacent FadA molecules. The leucine residues highlighted in green or cyan (residues 71, 78, 93 and 101 in sequence above) form Leu-Leu contacts stabilizing the 2 antiparallel helices of one monomer (L53-L84 and L60-L76, numbering on mFadA).

Intramolecular interactions are stabilised by interactions between Tyrosine (Tyr or Y) aromatic rings (Y50, Y73 and Y80, numbering on mFadA). Several Leu residues are involved in non-covalent interaction with other monomers (numbering on mFadA—SEQ ID NO:2, in pink in one monomer, underlined on the other one). This involves L7, 11, 14 and 21 interacting with L53, 76 and 84 (residue numbers are in the context of mFadA or SEQ ID NO:2).

Active Complex FadAc

Following the teaching of Temoin et al FEBS Lett. 586; 1-6 (2012), the FadAc, made up of full length of SEQ ID NO:1 was proposed as a vaccine candidate. As mentioned above, this reference showed that an inhibitory cell attachment assay showed that the pre-FadA-mFadA complex (FadAc) inhibited the attachment of F. nucleatum to host cells, while mFadA alone did not.

Cyto-mFadA

A further design was to mimic the functional FadAc complex and the component of this complex: mFadA (FadA sequence without its signal peptide). The following sequence was chosen for evaluation:

Cyto_mFadA: from residue 19 (after signal peptide) to 129 (end of the sequence). Underlined residues are heterologous residues to be able to express, purify and characterise the construct.

MANDAASLVGELQALDAEYONLANQEEARFNEERAQADAARQALAQNEQ
VYNELSQRAQRLQAEANTRFYKSQYQDLASKYEDALKKLESEMEQQKAI
ISDFEKIQALRAGNGGHHHHHH

Tip-FadA

A further design was performed in order to try to focus the raised immune response more against the tip part of the antigen which is the part responsible for binding to the host cell. The goal was to reduce the size of the mFadA prototype to its minimum around the tip hairpin but still allowing a native folding of the construct to present correctly the tip part to the immune system.

To define the limits of this shorter prototype, the tools used were: a 3D model of our protein sequence (based on the available 3D protein structure—PDB code 3TEW), the contact residue tool available in the Moleculart Operating Environment tool. The goal was to keep all residues allowing 3D structure stabilisation interactions.

For the C-terminal limit, it was decided to keep until Met109. This residue is predicted to be involved in the packing of the Tyr68 which is known to be essential for hydrophobic packing of the structure. For the N-terminal limit, the residue Gln66 is a good starting residue of an α-helix. It was decided to add a heterologous Pro residue which has the propertied to be a good α-helix enhancer. Moreover, an additional heterologous Alanine residue was added between the Pro and the first Met due to the N terminal Methionine processing phenomena that could be observed in E. coli when the second residue of the protein sequence is a Proline. By adding this residue, we want to ensure an homogenous protein population after expression.

The following sequence resulted:

Tip_FadA: from residue 66 to 109. Underlined residues are heterologous residues to be able to express, purify and characterise the construct.

MAPQVYNELSQRAQRLQAEANTRFYKSQYQDLASKYEDALKKLESEMGG
HHHHHH

### Example 2—Expression and Purification of FadA Proteins

Expression

Genes encoding full length FadA (to make FadAc), cyto_mFadA and Tip_Fad A proteins were codon-optimized, synthesised, and cloned into the pET24 expression plasmids by GENEWIZ. Final constructs were generated by the transformation of E. coli B834 (DE3) strain with the appropriate recombinant expression vectors. E. coli transformants were stripped from agar plate containing 50 μg/ml of kanamycin (Kan) and used to inoculate 800 ml of LB broth+kan (50 μg/ml) to obtain 00600 around 0.05. Cultures were incubated at 37° C. with agitation (300 rpm) until the OD₆₀₀ reach 0.6-0.8. At this time, flasks were placed on ice for one hour to cool down the culture before adding 1 mM of Isopropyl β-D-1-thiogalactopyranoside (IPTG) in each flask. Induction was performed O/N at 16° C. After overnight induction, cultures were centrifuged.

Cyto_mFadA and Tip_FadA Purification

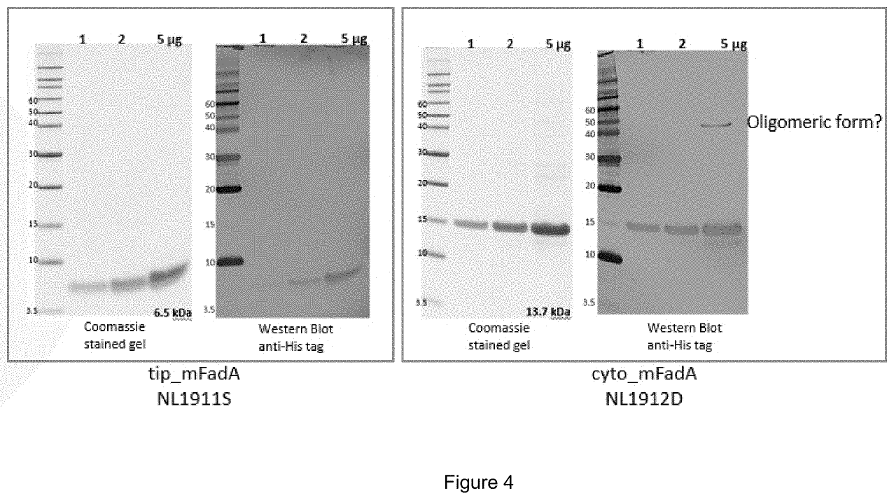

The pellets from the overnight culture were resuspended in lysis buffer (50 mM NaH₂PO₄, 0.3 M NaCl at pH 8.0) supplemented with complete protease inhibitor (Roche Applied Science, Indianapolis, IN). Cells were disrupted through one shot at 30 kPsi followed by one shot at 15 kPsi using Constant System and insoluble fraction was separated from soluble fraction through 15 min of centrifugation at 20 000×g, 4° C. The soluble fractions were then mixed with TALON (Clontech, Mountain View, CA) resin pre-equilibrated in lysis buffer and incubated at 4° C. for one hour with agitation to allow protein binding. The resin was washed with 10 c.v. of 50 mM NaH₂PO₄, 0.3 M NaCl pH 8.0 buffer, and then protein was eluted with 4 c.v. of 50 mM NaH₂PO₄, 0.3 M NaCl, 150 mM imidazole pH 8.0 buffer. Elution fraction was adjusted to 8M urea and pH to 4.0 and loaded into POROS 50 HS resin (GE Healthcare, Piscataway, NJ) pre-equilibrated with 8M urea, 0.3 M NaCl, 50 mM acetic acid buffer. The column was washed with 8M urea, 0.3 M NaCl, 50 mM acetic acid buffer followed by another wash with 8M urea, 1M NaCl, 50 mM acetic acid buffer. Fractions from the wash with 8M urea, 0.3 M NaCl, 50 mM acetic acid buffer were pooled together and dialysed against 50 mM phosphate pH 8.0 buffer. After dialysis, the proteins were sterile filtered and stored at −80° C. Protein purity was achieved by SDS-PAGE and the protein concentration by RC DC™ (reducing agent and detergent compatible) protein assay (Biorad, Hercules, CA). FIG. 4 shows the results of SDS-PAGE and Western blotting for the purified tip-mFadA and cyto-mFadA.

In contrast, when the full length of FadA (SEQ ID NO:1) was expressed and purified in the same way, the degree of multimerisation of the FadA led to difficulties in purification of FadA due to the gelatinous nature of the expressed protein. The same results were obtained even when different E. coli strains were used for expression.

### Example 3: Immunisation of Mice with mFadA in AS01 Formulation

Mouse Immunisation

Groups of 12 female Balb/C mice were immunized intramuscularly at days 0, 14 and 28 with 10 μg of Cyto-mFadA or 10 μg of Tip-mFadA, both adjuvanted with the adjuvant system 01 (AS01). (After formulation, the amount of protein immunised was 8.754). A control group of mice did receive the adjuvant alone.

Anti-mFadA and anti-living Fusobacterium nucleatum ELISA titres were determined in individual sera collected at day 42 (14 days post-Ill).

Anti-mFadA ELISA Response: Protocol

Microtiter plates were coated overnight at 4° C. with Cyto-mFadA at the concentration of 5 μg/ml in phosphate buffer saline (PBS), except one line which was coated with affinity purified goat anti-mouse IgG antibodies (Ref AP121 Millipore™). Plates were then blocked for 30 minutes with saturation buffer (bovine serum albumin—BSA—1% in PBS). After washing, two-fold serial dilutions of mouse sera (diluted in BSA 0.2%-Tween 20 0.05%-PBS) were added into mFadA coated wells while two-fold serial dilutions of a commercial calibrated IgG reference were added into anti-mouse IgG coated wells. The plates were incubated at 25° C. for 30 min with agitation. After washing, bound mouse antibodies were detected using peroxidase-conjugated goat anti-mouse IgG antibodies (Ref 115-035-033 Jackson). The detection antibodies were incubated for 30 min. at 25° C. with agitation. The colour was developed using 4 mg 0-phenylenediamine (OPD)+5 μl H₂O₂ per 10 ml pH 4.5 0.1M citrate buffer for 15 minutes in the dark at room temperature. The colorimetric reaction was stopped with HCl 1N and the optical density (OD) was read at 490 nm using a spectrophotometer for microtiter plates. The level of antigen-specific IgG antibodies was established by reporting the optical densities of the tested samples to the OD curve of the IgG reference and calculated by the 4-parameter method using the Soft Max Pro software.

Anti-Bacteria ELISA Response: Protocol

Microtiter plates were coated overnight at 37° C., until evaporation, with living F. nucleatum (ATCC 23726) in PBS. Plates were then blocked for 30 minutes with saturation buffer (BSA 1% in PBS). After washing, two-fold serial dilutions of mouse sera (diluted in BSA 0.2%-Tween 0.05%-PBS) were incubated at 37° C. for 60 min with agitation. After washing, bound mouse antibodies were detected using peroxidase-conjugated goat anti-mouse IgG antibodies (Ref 115-035-033 Jackson). The detection antibodies were incubated for 30 min. at 37° C. with agitation. The colour was developed using 4 mg OPD+5 μl H₂O₂ per 10 ml pH 4.5 0.1M citrate buffer for 15 minutes in the dark at room temperature. The colorimetric reaction was stopped with HCl 1N and the OD was read at 490 nm using a spectrophotometer for microtiter plates. The level of anti-heat inactivated bacteria was expressed in mid-point titers.

Assessment of the FadA Specificity of the Anti-Bacteria Response: Protocol

Pooled sera of mice immunized with Cyto-mFadA, Tip-mFadA or the adjuvant alone were incubated with 200 μg/mL of Cyto-mFadA for 1 hour at 37° C. under agitation, in order to deplete their anti-mFadA antibodies. They were then tested for their remaining anti-bacteria antibody content as described here above.

Results

Serum IgG responses against mFadA and living bacteria are shown in the Table 1. Cyto-mFadA induced strong IgG responses, whatever the ELISA, much higher than those elicited by Tip-mFadA.

TABLE 1
IgG response against mFadA and living bacteria

| | | |
|---|---|---|
| | Anti-mFadA | Anti-bacteria IgG |
| | IgG μg/ml | Mid-point titer |
| Vaccine formulation | Geometric mean (95% Cl) | Geometric mean (95% Cl) |

| | | | | |
|---|---|---|---|---|
| Tip-mFadA AS01 | 0.17 | (0.1-0.6) | 52.71 | (28.7-96.8) |
| Cyto-mFadA AS01 | 273.7 | (179-418.6) | 3693.9 | (2034-6708.5) |

| | | | |
|---|---|---|---|
| AS01 | 0.02 | 48.6 | (38.9-60.7) |

As shown in the Table 2, 87% of the anti-bacteria response generated by Cyto-mFadA was removed by pre-incubating the corresponding serum with mFadA, confirming that this response was mostly FadA-specific. Such an effect was not observed on the sera from the other groups.

TABLE 2
Anti-bacteria IgG response in sera pre-incubated with mFadA

| | | Anti-bacteria IgG in pooled | |
|---|---|---|---|
| | Anti-bacteria IgG | sera pre-incubated with | |
| | in untreated | mFadA | |
| | pooled sera | Mid-point titer (reduction % | |
| Vaccine formulation | Mid-point titer | versus untreated serum) | |
| Tip-mFadA AS01 | 86 | 118 | |
| Cyto-mFadA AS01 | 5232 | 676 | (87%) |
| AS01 | 72 | 63 | (13%) |

### Example 4—Induction of Abs in Gut of Mice Upon IM or Oral Immunization with Cyto mFadA

Groups of twelve 4 to 8 weeks old female Balb/c mice were immunized either intramuscularly (IM) or orally (IG) on days 0, 14 and 28. The IM groups were immunized with 10 μg of cyto mFadA formulated in an adjuvant whereas the oral delivery groups were immunized with 30 or 60 μg of cyto-mFadA adjuvanted with LT. Faeces were collected at day 42 and the level of specific anti-mFadA IgG was measured by ELISA, measuring the IgG that specifically bound to cyto-mFadA.

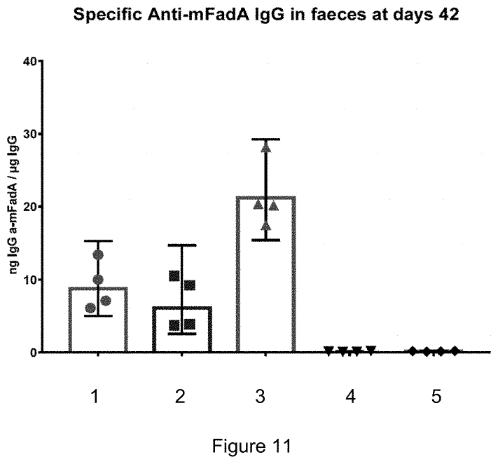

The results shown in FIG. 11 show that antigen specific anti-cyto mFadA IgG were detected in faeces at days 42 after immunization with 10 μg of adjuvanted cyto-mFadA, administered by IM route. No IgG response was detected in faeces from mice immunized orally with mFadA mixed with LT adjuvant, despite the use of higher antigen doses.

### Example 5—Bacteriophage Delivery in a Prime and Kill System

Demonstration of Expression of Antigen Following Recombinant Phage Transduction

Expression of a Protein (PE) Following Transduction of E. coli Culture with a Recombinant Bacteriophage Particle with a Recombinant Genome (Cosmid)

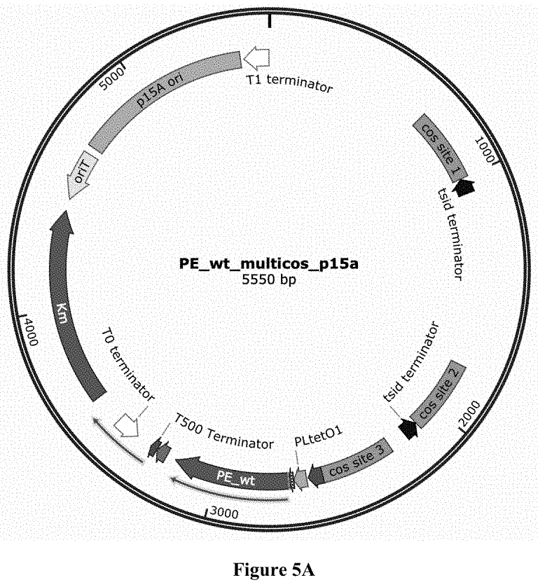

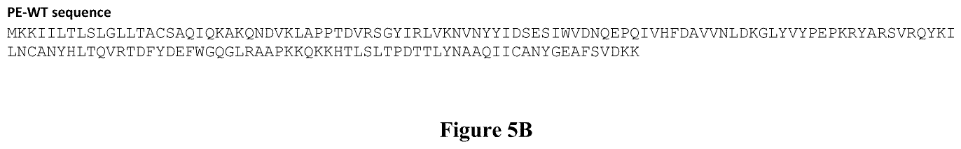

The cosmid shown in FIG. 5A was constructed including a gene encoding the PE protein shown in FIG. 5B, a kanamycin resistance gene, origins of replication (P15a) and cos sites to allow the packaging of the cosmid into phage capsids. Protein E (PE) is a ubiquitous antigen from non-encapsulated forms of Haemophilus influenzae (NTHi), described as important for the adhesion of the bacterium to epithelial cells (Ronander et al (2008) Microbes Infect 10:87-96. Protein E (PE) is known to bind to vitronectin, which protects the bacterium from complement attack (Singh et al (2013) Infect Immun 81:801-814.
E coli C2987: is a non-pathogenic E. coli strain
E. coli DH5a: is a non-pathogenic E. coli strain.
E. coli EMG2 ATCC® 23716: is a pathogenic E. coli strain.
E. coli Δrep: is a non-pathogenic E. coli strain. Loss of rep function blocks the life cycle of helper phage contamination.
KanR: kanamycin resistance
P2ᵥᵢᵣ₁: a mutant of bacteriophage P2 that is strictly lytic, used as a helper phage to provide P2 capsid proteins for the cosmid packaging.
P15a: origin of replication
Tet01 promoter: costitutive strong promoter in E coli. This promoter is repressed when the Z1 repressor is present (such as in DH5αZ1 strain). The addition of anhydrotetracycline hydrochloride (aTc) switches off the Z1 repressor and leads to strong expression in strains containing Z1 repressor.
Cos site: phage derived sequences essential for packaging into phage capsids.
Transducing particles: engineered bacteriophage with synthetic genomes.

The following experiment shows the ability of a bacteriophage to bind to and enter E. coli and to express a PE antigen under the control of a Tet01 promoter in E. coli.

Preparing Recombinant Bacteriophage:

To produce bacteriophage particles, 20 ml of LB containing 2 mM CaCl2) was seeded with 0.5 ml of an overnight culture of E coli C2987 containing PE wt cosmid that was grown at 37° C. in presence of kanamycin (50 μg/mL). The culture was grown at 37° C. to OD600 of 1. The culture was infected with 10 μl of P2ᵥᵢᵣ₁ (10¹¹) with MOI=0.1 and let to stand for 10 min at 37° C. The following reagents were added: CaCl₂ to a final concentration of 5 mM, MgCl₂ to a final concentration of 16 mM and glucose to a final concentration of 1%. The culture was shaken at 180-200 rpm at 37° C. for 1 hour and then EDTA was added to a final concentration of 10 mM. The culture was shaken at 250 rpm at 37° C. until lysis occurred (1-3 hours). 2 ml of chloroform was added and the mix was incubated at room temperature (RT) for 30 min whilst being shaken slowly. The mix was then centrifuged at 13000 rpm at RT for 10 minutes and the supernatant was collected and filtered through 0.2 μM membrane and stored at 4° C. The P2ᵥᵢᵣ₁ titer checked on E coli C2987 was ˜10¹¹-10¹².

Transduction with P2ᵥᵢᵣ₁ Lysate and Recombinant Bacteriophage Particles:

5 ml of an overnight culture of E coli C2987 cells were supplemented with CaCl₂ to a final concentration of 5 mM and were shaken at 37° C. for 15 minutes. 0.2 ml of the culture was taken and 0.2 ml of P2ᵥᵢᵣ₁ lysate including the recombinant bacteriophage particles (dilution for MOI=1) was added before incubating at 37° C. for 20 minutes with no shaking. 0.4 ml of 1M Na-citrate was added and mixed. 2.5-3 ml of Top agar 7 (TA7) pre-warmed at 42° C. were added, mixed and poured on the plate with Kanamycin (50 μg/mL). The plate was incubated overnight at 37° C. and Kanamycin resistant transductants were obtained.

Evaluation of the Expression of PE Antigen Following Transduction:

Ten E. coli C2987 transduced colonies were grown overnight at 30° C. in LB including Kanamycin (50 μg/mL). The OD600 of the overnight cultures was measured and they were diluted in 4% SDS in Tris pH6 according to the following formula: Volume of lysis buffer used to resuspend pellet from 1 mL of overnight culture (μL)=OD600/0.015 (This step is to normalise the number of cells from which protein is extracted). 1 mL of each culture was centrifuged at 12000 RCF. The resulting supernatants were discarded and the pellets were re-suspended and then heated to 95° C. for 10 minutes.

Dot blot was performed by spotting 2 μl of each sample onto nitrocellulose membrane and allowing the membrane to dry. As a positive control, 2 μl of 100 μg/ml PE antigen was used as presented in FIG. 2A. In FIG. 2B 2 μl of a negative control (non-transduced E. coli) was included in the dot blot. Non-specific binding was blocked by soaking the membrane in 5% BSA in TBS-T (20 mM Tris-HCl, 150 mM NaCl, 0.05% Tween 20, pH 7.5) for 30-60 minutes at room temperature. The membrane was then washed three times in TBS-T. The membrane was then incubated with a mouse monoclonal antibody against PE diluted in TBS-T containing 0.1% BSA for 30 minutes at room temperature. After washing three times in TBS-T, the membrane was incubated with an anti-mouse HRP conjugate for 30 minutes at room temperature. After washing three times in TBS-T and once with TBS, the membrane was incubated with ECL reagents and exposed to X-ray film to allow the detection of light. A Western blot was also performed to confirm the size of the expressed protein.

Results

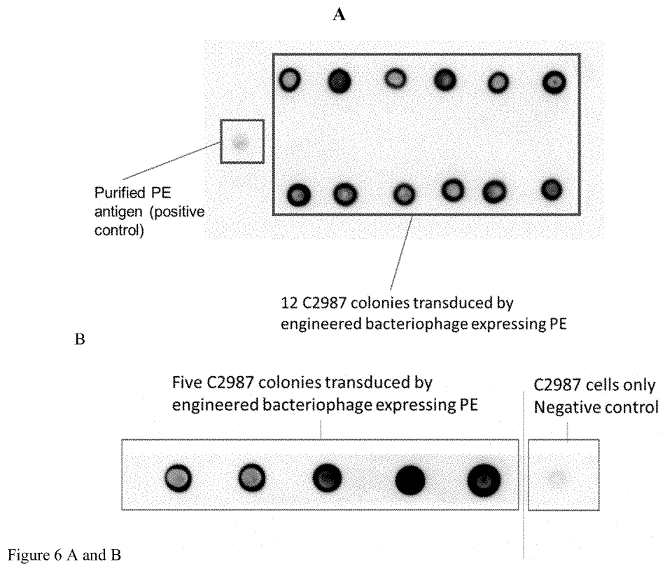

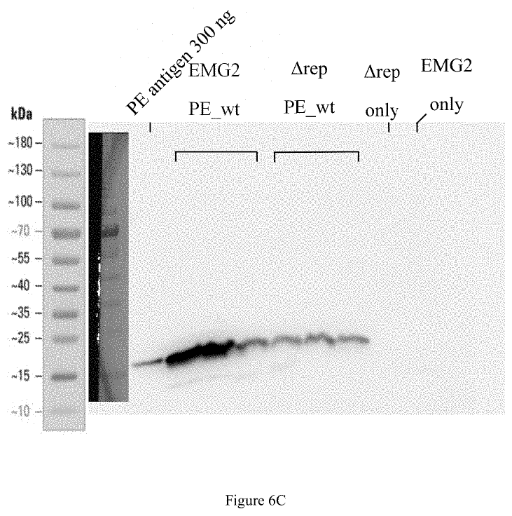

The results of the dot blot are shown in FIGS. 6A and B. As can be seen, all transduced E. coli colonies produced high levels of PE protein with darker spots being obtained in the transduced samples compared to the positive control which was loaded with 200 ng of PE protein.

FIG. 6C shows the results of a Western blot showing that the transduction particles were able to drive expression of the 20 kDa PE antigen in E. coli host cells. Strong bands are seen in the lines containing two strains of E. coli which have been transduced with bacteriophage carrying the PE gene under the control of a strong promoter. In the absence of bacteriophage transduction, no expression of PE is observed.

### Example 6 Expression of Lytic Activity in Bacteria Following Phage Transduction

The cosmid used for the expression of lytic activity is similar in design to the cosmid shown above for the expression of antigen, except the antigen encoding sequence was replaced with genes expressing bacteriophage lytic activity. Expression of the lytic activity was under the control of a Tet01 promoter that is repressed in DH5aZ1 E. coli cells in order to control lysis. The lytic activity is only expressed when the repressor is blocked by the addition of anhydrotetracycline hydrochloride.

The preparation of a bacteriophage was carried out as described above but using an E. coli DH5αZ1 production strain containing the cosmid encoding different lytic activities. The P2vir1 helper phage was used to infect the culture. Following lysis of the production strain, bacteriophage were collected from the medium. The medium contains some transducing particles which contain the cosmid as a surrogate genome and contaminating helper phage. The kanamycin resistance gene expressed from the cosmid allowed the selection of E. coli containing the cosmid and the presence of a Tet repressor in DH5αZ1 cells prevented the premature expression of lytic activity.

Assessment of Lytic Activity of Transduction Particles

Preparation of Engineered Bacteriophage Transduction Particles:

To produce bacteriophage transduction particles, 20 ml of LB containing 2 mM CaCl₂) was seeded with 0.5 ml of an overnight culture of E coli DH5αZ1 containing lysin containing cosmid that was grown at 37° C. in presence of kanamycin (50 μg/mL). The culture was grown at 37° C. to OD600 of 1. The culture was infected with 10 μl of P2ᵥᵢᵣ₁ (10¹¹) with MOI=0.1 and let to stand for 10 min at 37° C. The following reagents were added: CaCl₂ to a final concentration of 5 mM, MgCl₂ to a final concentration of 16 mM and glucose to a final concentration of 1%. The culture was shaken at 180-200 rpm at 37° C. for 1 hour and then EDTA was added to a final concentration of 10 mM. The culture was shaken at 250 rpm at 37° C. until lysis occurred (1-3 hours). 2 ml of chloroform was added and the mix was incubated at room temperature (RT) for 30 min whilst being shaken slowly. The mix was then centrifuged at 13000 rpm at RT for 10 minutes and the supernatant was collected and filtered through 0.2 μM membrane and stored at 4° C.

Transduction with P2ᵥᵢᵣ₁ Lysate and Bacteriophage Transduction Particles:

5 ml of an overnight culture of E. coli DH5αZ1 cells were supplemented with CaCl₂ to a final concentration of 5 mM and were shaken at 37° C. for 15 minutes. 0.2 ml of the culture was taken and 0.2 ml of P2ᵥᵢᵣ₁ lysate including the bacteriophage transducing particles (dilution for MOI=1) was added before incubating at 37° C. for 20 minutes with no shaking. 0.4 ml of 1M Na-citrate was added and mixed. 2.5-3 ml of Top agar 7 (TA7) pre-warmed at 42° C. were added, mixed and poured on the plate with Kanamycin (50 μg/mL). The plate was incubated overnight at 37° C. and Kanamycin resistant transductants were obtained.

Evaluation of the Lysis Following Transduction:

Several E. coli DH5αZ1 transduced colonies were grown overnight at 37° C. in LB including Kanamycin (50 μg/mL). Ten μL of the overnight culture was added to 2 ml LB supplemented with 50 μg/mL kanamycin. The refreshed cultures were grown at 37° C. for approximately 3 hours (yielding OD600 of approximately 0.1). Anhydrotetracycline hydrochloride (aTC) was added at different time points (t=0, 8 and/or 20 hours) to the cultures to allow for the induction lytic activity. Lysis was monitored as indicated by the loss of OD600 up to 24 hours.

Results

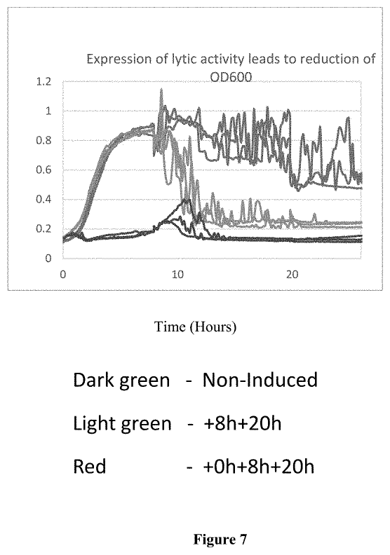

As shown in FIG. 7, in cultures where the lytic activity was not induced, the E. coli grew to an OD600 approaching 1.0 by 10 hours of culture and a high density was maintained up to 20 hours. Where lytic activity was induced after 8 hours, the culture also grew to density of OD600 of almost 1.0 at 10 hours but subsequent lysis of the E. coli reduced the OD600 to about 0.25 by 20 hours. Where the lytic activity was induced at 0 hours, 8 hours and 20 hours, the OD600 remained low at 0.2 at 10 hours indicating that lytic activity was killing the E. coli.

### Example 7 Co-Expression of Antigen and Lytic Activity after Transduction of E. coli

A cosmid was constructed in which two antigens (PE and green florescent protein) were under the control of a strong promoter and genes encoding lytic activity were under the control of a weaker promoter. DH5αZ1 E. coli cells were transduced with the combination cosmid and plated on kanamycin so that only E. coli containing the cosmid could grow. The expression of PE and green fluorescent protein were constitutive whereas expression of the lytic activity was induced after 0, 1 and 2 hours. The OD 600 of the culture was monitored to measure growth of the culture and the decrease in OD600 resulting from the induction of lytic activity. The expression of GFP was measured using fluorescence intensity.

In addition, the cultures were centrifuged at time points at which 50% or 100% of E. coli were lysed. Pellets and supernatants were collected for subsequent analysis. Part of the supernatants were also concentrated to provide samples that were used for Western blot analysis.

Results

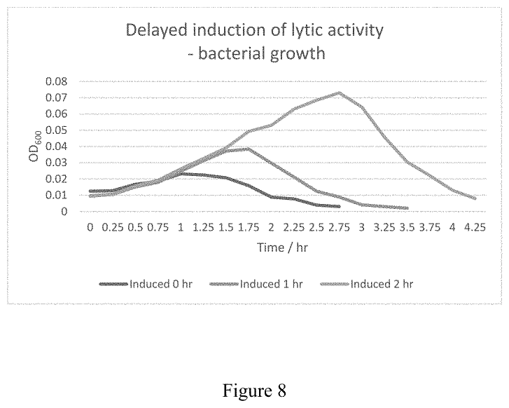

As shown in FIG. 8, where lytic activity is induced at time 0, little growth of the culture was observed. However, by delaying induction for 1 or 2 hours, more cell growth was achieved, followed by lysis and cell death. The expression of green fluorescent protein was monitored by following the fluorescence intensity of the cultures. As shown in FIG. 5, where lytic activity was induced at the start of the experiment, a little GFP was expressed with a fluorescence intensity of 1200 being achieved. Where lytic activity was induced after 1 hour, more GFP was expressed and a fluorescent intensity of 1,700 was achieved. The highest fluorescence intensity of over 3,000 was achieved where lytic activity was not induced until after 2 hours.

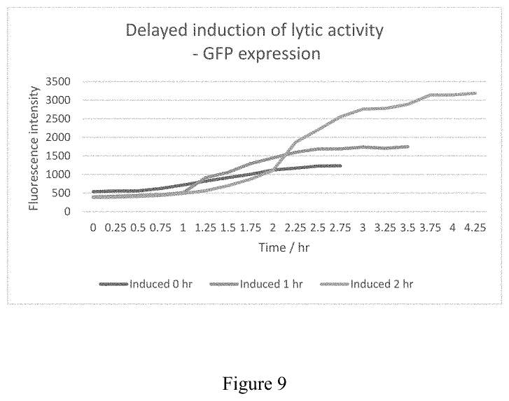

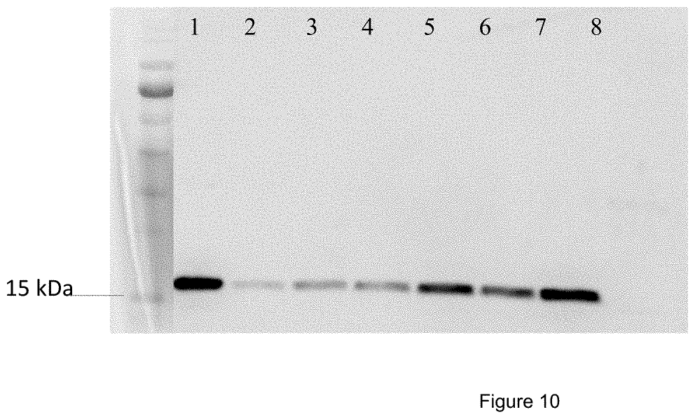

The expression of the PE antigen was monitored by Western blot of the concentrated supernatants and pellets collected in samples which had lytic activity induced after 0, 1 or 2 hours where the samples were collected at the point where 50% or 100% lysis had taken place. As shown in FIG. 9, PE could be detected in the concentrated supernatants with the highest levels being present where lytic activity was induced at 2 hours, however, even where lytic activity was induced at time 0, PE was detected in the supernatant. Moreover, as lysis progressed from 50% to 100% lysis, the antigen in the supernatant increases accordingly. Therefore, it is possible to make antigen in the transduced bacteria which is released on lysis of the bacteria

Western blot analysis of the samples showed that PE was detectable in the concentrated supernatant, as shown in the gel above) and also in the pellets. The presence of PE in the supernatant indicates that the delivery of a single cosmid (encoding PE, GFP and lytic activity) into E. coli, allowed the expression of PE antigen, and its subsequent release from the E. coli on lysis of the E. coli. Thus a bacteriophage containing a recombinant genome in which PE antigen expression is under the regulation of a strong promoter and lytic activity is under a weaker promoter allows the killing of the E. coli host and the release of antigen.

The results shown above demonstrate that it is possible to construct a cosmid encoding a lytic activity as well as one or more antigens. Such a cosmid can be delivered to bacteria in a transduction particle and is capable of both driving the expression of an antigen and lysing the host cell. This provides evidence that the prime and kill concept allows a dual attack approach for bacteriophage therapy in which a recombinant bacteriophage can both prime an immune response against selected antigens and kill the infected bacterium.

Sequences

Full length FadA F. nucleatum nucleatum
SEQ ID NO: 1
MKKFLLLAVLAVSASAFAANDAASLVGELQALDAEYQNLANQEEARFNEE
RAQADAARQALAQNEQVYNELSQRAQRLQAEANTRFYKSQYQDLASKYED
ALKKLESEMEQQKAIISDFEKIQALRAGN
mFadA F. nucleatum nucleatum
SEQ ID NO: 2
ANDAASLVGELQALDAEYQNLANQEEARFNEERAQADAARQALAQNEQVYNELSQRAQRLQAEANTRFYKSQY
QDLASKYEDALKKLESEMEQQKAIISDFEKIQALRAGN
mFadA with M and Histag F. nucleatum nucleatum
SEQ ID NO: 3
MANDAASLVGELQALDAEYQNLANQEEARFNEERAQADAARQALAQNEQVYNELSQRAQRLQAEANTRFYKSQ
YQDLASKYEDALKKLESEMEQQKAIISDFEKIQALRAGNGGHHHHHH
mFadA with M F. nucleatum nucleatum
SEQ ID NO: 4
MANDAASLVGELQALDAEYQNLANQEEARFNEERAQADAARQALAQNEQVYNELSQRAQRLQAEANTRFYKSQ
YQDLASKYEDALKKLESEMEQQKAIISDFEKIQALRAGN
Tip FadA F. nucleatum nucleatum
SEQ ID NO: 5
QVYNELSQRAQRLQAEANTRFYKSQYQDLASKYEDALKKLESEM
Tip FadA with MAP F. nucleatum nucleatum
SEQ ID NO: 6
MAPQVYNELSQRAQRLQAEANTRFYKSQYQDLASKYEDALKKLESEM
Tip FadA with MAP and Histag F. nucleatum nucleatum
SEQ ID NO: 7
MAPQVYNELSQRAQRLQAEANTRFYKSQYQDLASKYEDALKKLESEMGGHHHHHH
FadA signal peptide
SEQ ID NO: 8
MKKFLLLAVL AVSASAFA
Cyto_mFadA nucleotide with M and histag F. nucleatum nucleatum
SEQ ID NO: 9
ATGGCGAATGATGCCGCCAGTCTGGTTGGCGAACTGCAAGCGCTGGATGCCGAGTACCAGAATCTG
GCGAATCAAGAAGAGGCCCGCTTCAATGAAGAACGTGCCCAAGCGGATGCCGCCCGCCAAGCGCTG
GCCCAGAACGAGCAAGTGTACAACGAGCTCAGCCAACGTGCCCAACGTCTGCAAGCCGAGGCCAAT
ACCCGCTTCTACAAGAGTCAGTACCAAGATCTGGCCAGTAAGTACGAGGACGCGCTGAAGAAACTG
GAAAGCGAGATGGAGCAGCAGAAGGCGATCATCAGCGATTTCGAGAAGATCCAAGCCCTCCGCGCG
GGTAATGGCGGCCACCACCATCATCACCATTAATAA
Cyto_mFadA nucleotide with M F. nucleatum nucleatum
SEQ ID NO: 10
ATGGCGAATGATGCCGCCAGTCTGGTTGGCGAACTGCAAGCGCTGGATGCCGAGTACCAGAATCTG
GCGAATCAAGAAGAGGCCCGCTTCAATGAAGAACGTGCCCAAGCGGATGCCGCCCGCCAAGCGCTG
GCCCAGAACGAGCAAGTGTACAACGAGCTCAGCCAACGTGCCCAACGTCTGCAAGCCGAGGCCAAT
ACCCGCTTCTACAAGAGTCAGTACCAAGATCTGGCCAGTAAGTACGAGGACGCGCTGAAGAAACTG
GAAAGCGAGATGGAGCAGCAGAAGGCGATCATCAGCGATTTCGAGAAGATCCAAGCCCTCCGCGCG
GGTAATTAATAA
Cyto_tip_fadA nucleotide with MAP and histag F. nucleatum nucleatum
SEQ ID NO: 11
ATGGCCCCACAAGTGTACAACGAGCTCAGCCAACGTGCCCAACGTCTGCAAGCCGAGGCCAATACC
CGCTTCTACAAGAGTCAGTACCAAGATCTGGCCAGTAAGTACGAGGACGCGCTGAAGAAACTGGAA
AGCGAGATGGGCGGCCACCACCATCATCACCATTAATAA
Cyto_tip_fadA nucleotide MAP F. nucleatum nucleatum
SEQ ID NO: 12
ATGGCCCCACAAGTGTACAACGAGCTCAGCCAACGTGCCCAACGTCTGCAAGCCGAGGCCAATACC
CGCTTCTACAAGAGTCAGTACCAAGATCTGGCCAGTAAGTACGAGGACGCGCTGAAGAAACTGGAA
AGCGAGATGTAATAA
-PE sequence
SEQ ID NO: 13
MKKIILTLSLGLLTACSAQIQKAKQNDVKLAPPTDVRSGYIRLVKNVNYYIDSESIWVDNQEPQIV
HFDAVVNLDKGLYVYPEPKRYARSVRQYKILNCANYHLTQVRTDFYDEFWGQGLRAAPKKQKKHTL
SLTPDTTLYNAAQIICANYGEAFSVDKK
-Fusobacterium nucleatum animalis strain 11_3_2
SEQ ID NO: 14
MKKFLLLAVLAVSASAFAANDAASLVGELQALDAEYQNLANQEEARFNEEKAQADAAKQALAQNEQVYNE
LSQRAQRLQAEANTRFYKSQYQELASKYEDALKKLEAEMEQQKGVISDFEKIQALRAGN
-Fusobacterium nucleatum animalis strain 11_3_2 mFadA
SEQ ID NO: 15
ANDAASLVGELQALDAEYQNLANQEEARFNEEKAQADAAKQALAQNEQVYNE
LSQRAQRLQAEANTRFYKSQYQELASKYEDALKKLEAEMEQQKGVISDFEKIQALRAGN
-Fusobacterium nucleatum animalis strain 11_3_2 mFadA
SEQ ID NO: 16
MANDAASLVGELQALDAEYQNLANQEEARFNEEKAQADAAKQALAQNEQVYNE
LSQRAQRLQAEANTRFYKSQYQELASKYEDALKKLEAEMEQQKGVISDFEKIQALRAGN
-Fusobacterium nucleatum animalis strain 11_3_2 mFadA
SEQ ID NO: 17
MANDAASLVGELQALDAEYQNLANQEEARFNEEKAQADAAKQALAQNEQVYNE
LSQRAQRLQAEANTRFYKSQYQELASKYEDALKKLEAEMEQQKGVISDFEKIQALRAGNGGHHHHHH
-Fusobacterium nucleatum vincentii strain KCOM 2931
SEQ ID NO: 18
MKKFLLLAVLVVSASAFAANDAASLVGELQALDAEYQNLANQEEARFNEEKAQADAAKQALAQNEQVYNE
LSQRAQRLQAEANTRFYKSQYQDLASKYEDALKKLEAEMEQQKGVISDFEKIQALRAGN
-Fusobacterium nucleatum vincentii strain KCOM 2931
SEQ ID NO: 19
ANDAASLVGELQALDAEYQNLANQEEARFNEEKAQADAAKQALAQNEQVYNE
LSQRAQRLQAEANTRFYKSQYQDLASKYEDALKKLEAEMEQQKGVISDFEKIQALRAGN
-Fusobacterium nucleatum vincentii strain KCOM 2931
SEQ ID NO: 20
MANDAASLVGELQALDAEYQNLANQEEARFNEEKAQADAAKQALAQNEQVYNE
LSQRAQRLQAEANTRFYKSQYQDLASKYEDALKKLEAEMEQQKGVISDFEKIQALRAGN
-Fusobacterium nucleatum vincentii strain KCOM 2931
SEQ ID NO: 21
MANDAASLVGELQALDAEYQNLANQEEARFNEEKAQADAAKQALAQNEQVYNE
LSQRAQRLQAEANTRFYKSQYQDLASKYEDALKKLEAEMEQQKGVISDFEKIQALRAGNGGHHHHHH
-Fusobacterium nucleatum polymorphum strain 12230
SEQ ID NO: 22
MKKFLLLAVLAVSASAFAATDAASLVGELQALDAEYQNLANQEEARFNEERAQADAARQALAQNEQVYNE
LSQRAQRLQAEANTRFYKSQYQELASKYEDALKKLEAEMEQQKAVISDFEKIQALRAGN
-Fusobacterium nucleatum polymorphum strain 12230 mFAD
SEQ ID NO: 23
ATDAASLVGELQALDAEYQNLANQEEARFNEERAQADAARQALAQNEQVYNE
LSQRAQRLQAEANTRFYKSQYQELASKYEDALKKLEAEMEQQKAVISDFEKIQALRAGN
-Fusobacterium nucleatum polymorphum strain 12230 mFAD
SEQ ID NO: 24
MATDAASLVGELQALDAEYQNLANQEEARFNEERAQADAARQALAQNEQVYNE
LSQRAQRLQAEANTRFYKSQYQELASKYEDALKKLEAEMEQQKAVISDFEKIQALRAGN
-Fusobacterium nucleatum polymorphum strain 12230 mFAD
SEQ ID NO: 25
MATDAASLVGELQALDAEYQNLANQEEARFNEERAQADAARQALAQNEQVYNE
LSQRAQRLQAEANTRFYKSQYQELASKYEDALKKLEAEMEQQKAVISDFEKIQALRAGNGGHHHHHH
animalis
SEQ ID NO: 26
QVYNELSQRAQRLQAEANTRFYKSQYQELASKYEDALKKLEAEM
Fusobacterium nucleatum vincentii strain KCOM 2931
SEQ ID NO: 27
QVYNELSQRAQRLQAEANTRFYKSQYQDLASKYEDALKKLEAEM
Fusobacterium nucleatum polymorphum strain 12230
SEQ ID NO: 28
QVYNELSQRAQRLQAEANTRFYKSQYQELASKYEDALKKLEAEM

What is claimed is:

1.-15. (canceled)

16. A truncated Fusobacterium nucleatum Fusobacterium adhesin A (FadA) protein wherein at least a signal peptide which is at least 80%, 85%, 90% or 95% identical to SEQ ID NO:8 is deleted from the N-terminus of the FadA protein.

17. The truncated Fusobacterium nucleatum FadA protein of claim 16 having an amino acid sequence which is at least 80%, 85%, 90%, 95%, 97%, 98%, 99% or 100% identical to SEQ ID NO: 2-7 or 14-25.

18. The truncated FadA protein of claim 16 which is not associated with a further FadA molecule which contains a signal peptide at least 90% identical to MKKFLLLAVL AVSASAFA (SEQ ID NO:8).

19. The truncated FadA protein of claim 16 comprising a portion having an amino acid sequence at least 80% identity to SEQ ID NO:5, 26, 27 or 28.

20. The truncated FadA protein of claim 16 comprising at least 40, 45, 50, 55, 60, 70, 80, 90, 100 or 110 amino acids.

21. The truncated FadA protein of claim 16 which is capable of generating an immune response against F. nucleatum.

22. The truncated FadA protein of claim 21, wherein the FadA protein is capable of eliciting an immune response which is capable of preventing F. nucleatum from binding to a human cell.

23. The truncated FadA protein of claim 16 which is in an oligomeric form.

24. The truncated FadA protein of claim 23 wherein 2-20, 3-15, 4-10, 2-10 or 2-5 truncated FadA proteins are non-covalently bound together.

25. A polynucleotide encoding the truncated FadA of claim 16.

26. The polynucleotide of claim 25 having a nucleotide sequence at least 80%, 85%, 90%, 95%, 97%, 99% or 100% identical to SEQ ID NO:9, 10, 11 or 12.

27. A vector comprising the polynucleotide of claim 25 wherein the expression of FadA is under transcriptional control of a promoter.

28. A recombinant bacteriophage comprising a phage genome polynucleotide including a gene encoding the truncated FadA protein of claim 16.

29. A recombinant bacteriophage comprising a phage genome polynucleotide including a gene encoding the polynucleotide of claim 25.

30. The recombinant bacteriophage of claim 28 wherein the recombinant bacteriophage is adapted to bind to a host bacterium and insert the phage genome polynucleotide into said host bacterium.

31. The recombinant bacteriophage of claim 30 wherein the host bacterium is a Fusobacterium.

32. The recombinant bacteriophage of claim 31 wherein the host bacterium is a Fusobacterium nucleatum bacterium or E. coli.

33. A vaccine comprising the recombinant FadA of claim 16.

34. A recombinant FadA of claim 16 for use in therapy.

35. The recombinant FadA of claim 34 for use in the treatment or prevention of colorectal cancer in a human.
